SEQUENCE LISTING (15 sequences)

SEQ ID NO: 1 - DNA, 4244 bp, Bos Taurus
gccagtcgctgacagccgcagagctgagagcgtcttctctctcgcagaagcaggacttctgaatatatttgaaaactgaacagtttcaaccaagccgaagcatctgtcttcccagagacacaaatccaacttgagctgaatcacagcagatataagtcatcatggtgaaaagccacataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagtatctacctgcagccccgtagtggtggtgtctcatttcgtgcttctctctttgttacctgtatgctaatacccttggcgcttatagcactgggaaatgaagagcagacatgagatgctgtttattcaagtcccgttagctcagtatgctaatgccccatcttagcagtgattttgtagcaattttctcatttgtttcaagaacacgtgactacatttcccttttggaatagcatttctgccaagtctggaaggaggccacataatattcattcaaaaaaacaaaccggaaatccttagttcatagacccagggtccacctggttgagagcttgtgtcctgtgtctgcagagaactataaaggatattctgcattttgcaggttacatttgcaggtaacacagccagctattgcatcaagaatggatattcatgcaacctttgacttatgggtagaggacattttcacaaggaatgaacataatacgaaaggcttctgagactaaaaaattccaacatatgggagaggtgcccttggtggcagccttccattttgtatgtttaaagcaccttcaagtggtattcctttctttagtaacaaagtatagataattaagttaccttaatttaattaaactaccttctagacactgagagcaaatctgttgtttatctggaacccaggatgattttgacattgtttagagatgtgagagttgaactgtaaagaaagctgagtgctgaagaattgatgcttttgaactctagtgttggagaaaacttgagagtcccttggactgcaaggagatcaaattagtccatcctaaaggagatcagtcctgaatattcattggaaggactgatgctgaacgtgaaactccaatactttggccacctgatgggaagaactgaaggcaggaggagaaggggatgacagaggatgaagatggctggatggcatcatggattcaatggacatgagcttgagtaaactccaggagttggcaatcgacggagtcctggcatcctgcagtccatggtgtcgcagagttggacacgactgagtgactgaactgaggtgaacccagattttaacatagagaatgcagatataaaaactccatattcatttgattgaatcttttccttaaccagtgctagtgttggactggtaagattataacaacaaatataggttatgtgatgaagagaatagtgtacaaagaaaagaaatatgtgcatttctttattgctatcataattgtcaaaaaacaaaattaggtccttggtttctgtaaaattaacttttgaatcaacagggaggcatttaaagaaatatcttaaattagagacagtagaaatctgatacattcagagtggaaaaagaaattctattacgattatttaagaaggtaaaattatttcctgggttgttcaatattgtcacctagcagatagacactattgttctgcactgttattactggcttgcactttgtggtatcctatgtaaaaatacatatattgcatatgacagacttaagaatttctgttagagcaattaacatctgaactatctaatgcattacctgtttttgtaaggtactttttgtaaggtactaaggagacgtgggtttaatccctaggtcatgtaaatcccctggaggaggaaatagcaacccactccagtattcttgccaggagaatcccatgggcagaggagcctggcagggtgcagtccatgcatagggttgcaaagagtcagacaagacttgagctactaaacaataacaacaataaatgctgggttggctaaaaggttcattaggttttttttctgtaagatggctgtctttaacttcattcgaaacaattttgttagattgtatgtgacagctcttgtatcagcatgcatttgaaaaagaaaacaacttaccaaaattggtgaatttttgtatagccattttactattgaagatggaagaaaagaagcaaaattttcagcatatcatgctgtattatttcaagaaagataacacaaccaaaatgcgaaaatgtatttgtgcagtgtatggagaaggtgctgcaactgatcaagcttgtcaaagtagtttgtgaagtattgtgctggagatttcttactggacaatgctccacagtcgggtataccagttgaagttgatagtgatcaaattgagatattgagaacaatcaatgttataccacgtgggagatagctgacatactcaaaatatccaaatagaaccttgaaaaccatttgcaccatctcagttatgttaataactttgatgtttgagttccacataaattaagcaaaaaaaaaacaaaaacaaaaacacacaaccttgaccatatttgcatatgcagttctctactgaaatgaatgaaaacacttttgtttttaaaaacagattttgatgaacagtggatactatacaataacgtagaatggaaaagactgtggggtgagcaaaatgaaccagcaccaccaaaggccaggcttcatccaaagaagatgtgtgtatggtgggattggaaagtaatcctctattatgggattcttctggaaaaccaaaaaatcaattccaacaagtactgctcctaattagaccaactgaaagcagcattcaatgaaaagcatccagaattagtcaatagaaagcatataatcttccatcaggataacacaagactacatttctttgatgacccagcatggctgagaggttctgattcacctgctgtattcagacattgcatctttggatttccatttatttcagtctacagaattatcatcatgaaaaaaatttccattccctggaagattgtaaagtgcatctggaaaacttctttgctcaaaaagataaaaagttttgtgaacacagaattatgaagttgcctgaaaaacagcagaagatagtgactatgttgttcagtaaagttcttggtgcaaatgtgtcttttatttttatttaaacactaaaggcacgttttggccaacccaatactgaatacttaaaggaaactcttccgtgttgtccttagccttacagcgtgcactgaatagttttgtataagaatccagagtgatatttgaaatacgcatgtgcttatattttctatatttgtaactttgcatgtacttgttttgtgttaaaagtttataaatatttaatatctgactaaaattaaacaggagctaaaaggagg

SEQ ID NO: 2 - protein, 264 aa, Bos Taurus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 3 - DNA, 16 bp, Artificial Sequence
  Stopper sequence that introduces stop codon in 3 reading frames of target sequence
actagttaactgatca

SEQ ID NO: 4 - DNA, 753 bp, Saccharomyces Cerevisiae
gcaatgtcagacgcttgatggtaggataataataattccaaaaaaccatcataagacattcccaatgacagttgaaggtgagtttgccgcaaaacgcttcatagaagaaatggagcgctctaaaggagaatatttcaactttgacattgaagttagagatttggattatcttgatgctcaattgagaatttctagctgcataagatttggtccagtactcgcaggaaatggtgttttatctaaatttctcactggacgtagtgaccttgtaactcctgctgtaaaaagtatggcttggatgcttggtctgtggttaggtgacagtacaacaaaagagccagaaatctcagtagatagcttggatcctaagctaatggagagtttaagagaaaatgcgaaaatctggggtctctaccttacggtttgtgacgatcacgttccgctacgtgccaaacatgtaaggcttcattatggagatggtccagatgaaaacaggaagacaaggaatttgaggaaaaataatccattctggaaagctgtcacaattttaaagtttaaaagggatcttgatggagagaagcaaatccctgaatttatgtacggcgagcatatagaagttcgtgaagcattcttagccggcttgatcgactcagatgggtacgttgtgaaaaagggcgaaggccctgaatcttataaaatagcaattcaaactgtttattcatccattatggacggaattgtccatatttcaag

SEQ ID NO: 5 - DNA, 587 bp, Flavobacterium Okeanokoites
caactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaactt

SEQ ID NO: 6 - DNA, 27 bp, Artificial Sequence
  Zinc finger binding domain with 6 bp spacer at 10 to 15
rnnrnnrnnrnnnnnnnnnnynnynny

SEQ ID NO: 7 - DNA, 18 bp, Artificial Sequence
  Six finger zinc finger protein binding domain
rnnrnnrnnrnnrnnrnn

SEQ ID NO: 8 - DNA, 291 bp, Artificial Sequence
  Sequence encoding consensus zinc finger framework
ctcgagcccggggagaagccctatgcttgtccggaatgtggtaagtccttcagtaggaaggattcgcttgtgaggcaccagcgtacccacacgggtgaaaaaccatataaatgcccagagtgcggcaaatcttttagtcagtcgggggatcttaggcgtcatcaacgcactcatactggcgagaagccatacaaatgtccggaatgtggcaagtctttctcggattgtcgtgatcttgcgaggcaccaacgtactcacaccggtactagttaagtcgacgaggaggaggag

SEQ ID NO: 9 - DNA, 291 bp, Artificial Sequence
  Sequence encoding consensus zinc finger framework
gagctcgggcccctcttcgggatacgaacaggccttacaccattcaggaagtcatccttcctaagcgaacactccgtggtcgcatgggtgtgcccactttttggtatatttacgggtctcacgccgtttagaaaatcagtcagccccctagaatccgcagtagttgcgtgagtatgaccgctcttcggtatgtttacaggccttacaccgttcagaaagagcctaacagcactagaacgctccgtggttgcatgagtgtggccatgatcaattcagctgctcctcctcctc

SEQ ID NO: 10 - protein, 90 aa, Artificial Sequence
  Consensus zinc finger framework sequence
LEPGEKPYACPECGKSFSRKDSLVRHQRTHTGEKPYKCPECGKSFSQSGDLRRHQRTHTGEKPYKCPECGKSFSDCRDLARHQRTHTGTS

SEQ ID NO: 11 - protein, 5 aa, Artificial Sequence
  Zinc Finger protein consensus linker sequence
TGEKP

SEQ ID NO: 12 - DNA, 94 bp, Artificial Sequence
  PCR Primer
ctcgagcccggggagaagccctatgcttgtccggaatgtggtaagtccttcagtaggaaggattcgcttgtgaggcaccagcgtacccacacgg

SEQ ID NO: 13 - DNA, 84 bp, Artificial Sequence
  PCR Primer
acgcctaagatcccccgactgactaaaagatttgccgcactctgggcatttatatggtttttcacccgtgtgggtacgctggtg

SEQ ID NO: 14 - DNA, 94 bp, Artificial Sequence
  PCR Primer
cttttagtcagtcgggggatcttaggcgtcatcaacgcactcatactggcgagaagccatacaaatgtccggaatgtggcaagtctttctcgga

SEQ ID NO: 15 - DNA, 91 bp, Artificial Sequence
  PCR Primer
ctcctcctcctcgtcgacttaactagtaccggtgtgagtacgttggtgcctcgcaagatcacgacaatccgagaaagacttgccacattcc

(19) United States
(12) Patent Application Publication
(10) Pub. No.: US 2003/0051264 A1
(43) Pub. Date: Mar. 13, 2003

(54) Genetically modified cows having reduced susceptibility to mad cow disease

(76) Inventors: Monika Liljedahl, La Jolla, CA (US); Simon Eric Aspland, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/209,194

(22) Filed: Jul. 29, 2002

Related U.S. Application Data
(60) Provisional application No. 60/309,222, filed on Jul. 31, 2001.
(60) Provisional application No. 60/367,091, filed on Mar. 21, 2002.

(51) Int. Cl. A01K 67/027; C12N 5/06

(52) U.S. Cl. 800/15; 435/325

(57) ABSTRACT

The present invention relates to cow cells in which a gene associated with mad cow disease has been modified to reduce susceptibility to mad cow disease, cows having reduced susceptibility to mad cow disease, nucleic acids for making such cells and cows, and products obtained from such cows. The invention also includes methods of making each of the foregoing.

### CROSS REFERENCE TO RELATED APPLICATIONS

This application claims priority to U.S. Provisional Application Serial No. 60/309,222, filed Jul. 31, 2001, and U.S. Provisional Application Serial No. 60/367,091, filed Mar. 21, 2002; each of which is entitled GENETICALLY MODIFIED COWS HAVING REDUCED SUSCEPTIBILITY TO MAD COW DISEASE. The disclosures of each of the above Provisional Applications are hereby incorporated by reference in their entireties.

### BACKGROUND OF THE INVENTION

The prion diseases are a group of closely related transmissible diseases that affect the nervous system in both humans and animals. These include familial Creutzfeldt-Jakob disease (fCJD) and the novel prion disease, variant CJD (vCJD) in humans, bovine spongiform encephalopathy (BSE) and scrapie in sheep. Other animals are also known to carry the disease.

The prions are composed of abnormal isoforms of a host-encoded prion glycoprotein. Prion disease propagates by recruiting host cellular prion protein, composed primarily of α-helical structures, and transforming these into the disease-specific isoform rich in β-sheet structure. It has been proposed that PrPsc (the pathological isoform) acts as a template that promotes the conversion of PrPc (the non-pathological isoform) into PrPsc. There can be a mixture of different isoforms, and these different conformations encode different strains.

With the appearance of the novel human prion disease vCJD and the clear experimental evidence that it is caused by exposure to BSE, which has caused many people's death and made it necessary to kill off hundreds of thousands of cattle, it is very important to eradicate this disease.

The classic example of prion disease is scrapie, which occurs naturally in sheep. Recently familial Creutzfeldt-Jakob disease (fCJD), Gerstmann-Straussler syndrome (GSS), fatal familial insomnia (FFI) and also Kuru have received tremendous attention recently due to the epidemic of the Bovine Spongiform Encephalitis (BSE) that spreads to humans to cause variant Creuzfeldt-Jacob disease (vCJD).

fCJD, GSS, FFI and Kuru can be divided into three etiological categories: sporadic, acquired and inherited. Acquired prion disease includes iatrogenic CJD and Kuru, which arise from accidental exposure to human prions through medical or surgical procedures or participation in cannibalistic feasts. There is no evidence of association between scrapie and fCJD in humans (Brown et al., 1987). Sporadic CJD occurs in all countries in about one per million per year. Around 15% of human prion disease are inherited, and all cases to date have been associated with coding mutations in the prion protein gene (PRNP). There are 20 distinct types (Collinge, 1997). No pathogenic mutation is present in sporadic and acquired disease. However, a common PrP polymorphism at residue 129, where either methionine or valine can be encoded, is a key determinant of genetic susceptibility to acquired and sporadic prion disease. Mostly homozygous individuals (Collinge et al., 1991; Palmer et al., 1991; Windl et al., 1996) are affected.

The nature of the transmissible agent was obscure for many years. It was thought to be a “slow-virus,” but in 1967 Griffith suggested it to be a protein. In 1982, Bolton isolated a protease-resistant sialoglycoprotein, designated the prion protein (PrP). The name was proposed by Prusiner in 1982 from the first letter of proteinacious infectious particle. The protein accumulates in affected brains and sometimes causes amyloid deposits.

The protease-resistant PrP extracted from the brain is 27-30 kDa and is designated PrPsc (denoting the scrapie isoform), which is derived from a larger molecule of 30-33 kDa. The normal product of the gene is protease-sensitive and designated PrPc (cellular isoform of the protein). No difference in the amino acid sequence between PrPc and PrPsc have been identified. PrPsc is known to be derived from PrPc by a pottranslational modification (Borchelt et al., 1990; Caughey and Raymon, 1991).

In recent years in the United Kingdom, a novel human prion disease (vCJD) appeared which proved to be transmitted from cattle (BSE). It has caused a threat of a major epidemic not only in the UK but also in other countries as a result of dietary or other exposure to BSE prions (Wilesmith et al., 1988; Andersson et al., 1996). vCJD has a clinical presentation in which behavioral and psychiatric disturbances predominate. It is not unusual that patients first are referred to a psychiatrist due to depression, anxiety or behavioral symptoms. Other features include delusions, emotional liability, aggression, insomnia and auditory and visual hallucinations. The disease further progresses with cerebellar symptoms such as ataxia. Dementia usually develops later in the clinical course. The age of onset ranges from 16-51 years (mean 29 years) and the clinical course is 9-35 months (mean 14 months). vCJD can be diagnosed by detection of characteristic PrP immunostaining and PrPsc on tonsil biopsy. The presence of PrPsc on tonsils and lymphoreticular tissue is specific to vCJD and not present in other prion diseases (Collinge et al., 1997; Hill et al., 1999).

The neuropathological appearances of vCJD are very consistent. There is widespread spongiform changes, gliosis and neural loss, but the most remarkable is the abundant PrP amyloid plaques in cerebral and cerebellar cortex.

All cases are homozygous for methionine PRNP 129 and have one prion strain. This may explain why vCJD has a relatively stereotypic clinical presentation and neuropathology as compared to sporadic CJD.

Prion knock-outs have been achieved in mice. These animals cannot be infected with the prion disease. If the gene is reintroduced, the mice again become susceptible to the disease. The mice with the prion gene knocked out appear healthy and have no gross phenotype (Bueler et al., 1992) all through their life. This would argue that animals can have a normal life without the prion protein.

However, some groups have argued that in in vitro experiments the mice would have abnormal synaptic physiology (Collinge et al., 1992), whereas other groups have failed to reproduce these results (Lledo et al., 1996; Herms, 1995). These changes, if existing, do not seem to affect the health or life span of the mice devoid of the prion protein.

vCJD has been linked directly to eating meat from cattle infected with Bovine Spongiform Encephalopathy, which is commonly called “mad cow disease.” At least 100 people in Europe have died so far of vCJD since the mid-1990s.

About 1 million contaminated cattle may have entered the human food chain, and the future number of vCJD cases could range from 100 to 150,000 depending on the incubation period of BSE in humans. However, this risk is difficult to assess, because it largely depends on factors such as the virulence of the BSE agent adapted to primates and the efficiency of secondary transmission to humans.

Although BSE has mainly affected the UK since 1999, other countries in Europe besides the UK have reported confirmed cases of BSE. These include Belgium, Denmark, France, Germany, Ireland, Italy, Liechtenstein (in 1998), Luxembourg, the Netherlands, Portugal, Spain and Switzerland.

An endless number of cattle have now been slaughtered not only in the UK but also in other European countries. This is catastrophic for the cattle industry and shortage of meat is not only a concern in Europe but is also starting to become a problem in the U.S.

Various methods have been proposed to control the spread of BSE. Extensive tests have shown that BSE very rarely shows up in cattle under 30 months of age. In the UK since 1996, any cattle older than 30 months have been banned from sale as food for humans. Also, by law in the UK, the parts of cattle and sheep most likely to carry BSE must be removed. These parts are known as Specified Risk Material and include brain tissue and spinal cord. Further, since August 1996, there has been a ban in the UK on feeding farm animals (cattle, sheep, pigs and chickens) food derived from other mammals. Another approach is to screen cows, meat and also human blood that is to be used for blood transfusions.

Although the prion disease is quite well understood among other neurological degenerative diseases, a rational treatment has not yet been found. One approach is to find compounds that bind PrPsc, such as Congo red (Ingrosso et al., 1995) and polyene antibiotics (Pocchiari et al., 1987). Another is to use peptides that break the β-sheets (Soto et al., 2000). These strategies seem to have limited effects in animal models and have more significant effects if administered before clinical onset of disease, which is impractical because of toxicity.

The molecular event that causes the conformational change of PrPc to PrPsc still remains obscure. Any ligand that selectively stabilizes the PrPc state will prevent rearrangement and might block prion replication.

### SUMMARY OF THE INVENTION

One embodiment of the present invention relates to a method of obtaining a cow with reduced susceptibility to mad cow disease. The method can include obtaining a cell from a cow, generating a modified cell by modifying a gene in the cell which is associated with mad cow disease such that the modified gene provides reduced susceptibility to mad cow disease relative to an unmodified gene, and generating a cow from the modified cell, wherein the cow includes cells in which the gene associated with susceptibility to mad cow disease has been modified. The cell can be a somatic cell. The somatic cell can be, for example a fibroblast, a granulosa cell, fetal fibroblast and the like. Also, the cell can be a germ cell, a stem cell, any fetal cell and the like. The gene can be modified by replacing both chromosomal copies of said gene or a portion thereof with a homologous sequence which provides reduced susceptibility to mad cow disease. The homologous sequence can include a modified version of a gene which encodes an mRNA corresponding to the cDNA nucleotide sequence of SEQ ID NO: 1 or a portion of such a gene wherein the gene or portion thereof contains a stop codon in the open reading frame which encodes the polypeptide of SEQ ID NO: 2. The homologous sequence can include a modified version of a gene which encodes an mRNA corresponding to the cDNA nucleotide sequence of SEQ ID NO: 1 or a portion of such a gene wherein the gene or portion thereof contains a deletion therein. The gene can encode a polypeptide comprising the amino acid sequence of SEQ ID NO: 2. The methionine at position 129 of SEQ ID NO: 2 can be replaced with an amino acid other than methionine. The methionine at position 129 can be replaced by an amino acid including alanine, valine, cysteine, isoleucine, leucine, phenylalanine, tryptophane, tyrosine and the like. The homologous sequence can include a modified version of a cow prion gene which contains a stop codon in the open reading frame which encodes the cow prion polypeptide. The homologous sequence can include a modified version of a cow prion gene which contains a deletion therein. The gene can encode a polypeptide comprising the amino acid sequence of a cow prion polypeptide. The modified cell can be generated by replacing the gene encoding the cow prion protein or a portion thereof with a gene encoding a prion protein from a species other than cow or a portion thereof. The species can include a sheep, a goat, a marsupial, a mouse, and the like.

Also, another embodiment of the invention relates to a genetically engineered cow cell in which a gene associated with mad cow disease has been modified to provide reduced susceptibility to mad cow disease. The cell can be a skin fibroblast, a granulosa cell, a stem cell, a germ cell, a fetal fibroblast, any fetal cell and the like. Both chromosomal copies of the gene or portions thereof can be replaced with a homologous sequence which provides reduced susceptibility to mad cow disease. The homologous sequence can include a modified version of a gene which encodes an mRNA corresponding to the cDNA nucleotide sequence of SEQ ID NO: 1 or a portion of such a gene wherein the gene or portion thereof contains a stop codon in the open reading frame which encodes the polypeptide of SEQ ID NO: 2. The homologous sequence can include a modified version of a gene which encodes an mRNA corresponding to the cDNA nucleotide sequence of SEQ ID NO: 1 or a portion of such a gene wherein the gene or portion thereof contains a deletion therein. The gene can encode a polypeptide comprising the amino acid sequence of SEQ ID NO: 2. The methionine at position 129 of SEQ ID NO: 2 can be replaced with an amino acid other than methionine. The methionine at position 129 can be replaced by alanine, valine, cysteine, isoleucine, leucine, phenylalanine, tryptophane, tyrosine, and the like. The homologous sequence can include a modified version of a cow prion gene which contains a stop codon in the open reading frame which encodes the cow prion polypeptide. The homologous sequence can include a modified version of a cow prion gene which contains a deletion therein. The gene can encode a polypeptide including the amino acid sequence of a cow prion polypeptide. The modified cell can be generated by replacing the gene encoding the cow prion protein or a portion thereof with a gene encoding a prion protein from a species other than cow or a portion thereof. The species can be a sheep, a goat, a marsupial, a mouse, and the like.

Further, embodiments of the present invention relate to a recombinant nucleic acid that can include a 540 region homologous to a portion of a gene associated with susceptibility to mad cow disease, a 3′ region homologous to a portion of a gene associated with susceptibility to mad cow disease, and at least a portion of the coding sequence of the gene can be disposed between the 5′ region and the 3′ region, and the at least a portion of the coding sequence can contain a sequence therein which reduces susceptibility to mad cow disease. The sequence can include a modified version of a gene which encodes an mRNA corresponding to the cDNA nucleotide sequence of SEQ ID NO: 1 or a portion of such a gene wherein the gene or portion thereof contains a stop codon in the open reading frame which encodes the polypeptide of SEQ ID NO: 2. The sequence can include a modified version of a gene which encodes an mRNA corresponding to the cDNA nucleotide sequence of SEQ ID NO: 1 or a portion of such a gene wherein the gene or portion thereof contains a deletion therein. The gene can encode a polypeptide that includes the amino acid sequence of SEQ ID NO: 2. The methionine at position 129 of SEQ ID NO: 2 can be replaced with an amino acid other than methionine. The methionine at position 129 can be replaced by alanine, valine, cysteine, isoleucine, leucine, phenylalanine, tryptophane, tyrosine and the like. The sequence can include a modified version of a cow prion gene which contains a stop codon in the open reading frame which encodes the cow prion polypeptide. The sequence can include a modified version of a cow prion gene which contains a deletion therein. The gene can encode a polypeptide including the amino acid sequence of a cow prion polypeptide. The sequence can include a gene encoding a prion protein from a species other than cow. The species can be, for example, a sheep, a goat, a marsupial, a mouse, and the like. The recombinant nucleic acid can further include at least one nucleic acid encoding a detectable polypeptide and the at least one nucleic acid can be operably linked to a promoter. The detectable polypeptide can be CD8, CD4, green fluorescent protein, and the like. The recombinant nucleic acid can include a nucleic acid encoding CD8 or CD4 operably linked to a promoter and a nucleic acid encoding green fluorescent protein operably linked to a promoter, for example. At least one nucleic acid encoding a detectable polypeptide can be flanked by a site which facilitates recombination. The site which facilitates recombination can be, for example, a Lox P site and the like.

Another embodiment of the present invention relates to a genetically modified cow generated from the recombinant cell as described more fully herein. A cow in which a gene associated with mad cow disease or a portion thereof has been replaced with a sequence which reduces susceptibility to mad cow disease. Both chromosomal copies of the gene or a portion thereof can be replaced with a homologous sequence which provides reduced susceptibility to mad cow disease. The homologous sequence can include a modified version of a gene which encodes an mRNA corresponding to the cDNA nucleotide sequence of SEQ ID NO: 1 or a portion of such a gene wherein the gene or portion thereof contains a stop codon in the open reading frame which encodes the polypeptide of SEQ ID NO: 2. The homologous sequence can include a modified version of a gene which encodes an mRNA corresponding to the cDNA nucleotide sequence of SEQ ID NO: 1 or a portion of such a gene wherein the gene or portion thereof contains a deletion therein. The gene can encode a polypeptide that includes the amino acid sequence of SEQ ID NO: 2. The methionine at position 129 of SEQ ID NO: 2 can be replaced with an amino acid other than methionine. The methionine at position 129 can be replaced by alanine, valine, cysteine, isoleucine, leucine, phenylalanine, tryptophane, tyrosine, and the like. The homologous sequence can include a modified version of a prion gene which contains a stop codon in the open reading frame which encodes the polypeptide of the cow prion polypeptide. The homologous sequence can include a modified version of a cow prion gene which contains a deletion therein. The gene can encode a polypeptide that includes the amino acid sequence of cow prion polypeptide. The gene encoding the cow prion protein or a portion thereof can be replaced with a gene encoding a prion protein from a species other than cow or a portion thereof. The species can be a sheep, a goat, a marsupial, a mouse, and the like.

Further, embodiments of the present invention relate to a method of modifying a gene associated with susceptibility to mad cow disease. The method can include introducing a nucleic acid that includes a sequence homologous to at least a portion of the coding region of the gene into a cow cell, wherein the homologous sequence includes a sequence which reduces susceptibility to mad cow disease, and replacing at least one chromosomal copy of the gene with the homologous sequence. The sequence can include a modified version of a gene which encodes an mRNA corresponding to the cDNA nucleotide sequence of SEQ ID NO: 1 or a portion of such a gene wherein the gene or portion thereof contains a stop codon in the open reading frame which encodes the polypeptide of SEQ ID NO: 2. The sequence can include a modified version of a gene which encodes an mRNA corresponding to the cDNA nucleotide sequence of SEQ ID NO: 1 or a portion of such a gene wherein the gene or portion thereof contains a deletion therein. The gene can encode a polypeptide comprising the amino acid sequence of SEQ ID NO: 2. The methionine at position 129 of SEQ ID NO: 2 can be replaced with an amino acid other than methionine. The methionine at position 129 can be replaced by alanine, valine, cysteine, isoleucine, leucine, phenylalanine, tryptophane, tyrosine, and the like. The sequence can include a modified version of a cow prion gene which contains a stop codon in the open reading frame which encodes the polypeptide of the cow prion polypeptide. The sequence can include a modified version of a cow prion gene which contains a deletion therein. The gene can encode a polypeptide that includes the amino acid sequence of a cow prion polypeptide. The sequence can include a gene encoding a prion protein from a species other than cow. The species can be a sheep, a goat, a marsupial, a mouse, and the like. The methods can further include enhancing the rate of recombination by introducing double stranded break in the nucleic acid in a region in the vicinity of the gene associated with susceptibility to mad cow disease. For example the double stranded break can be induced by using at least one zinc finger endonuclease protein. Further, the methods can include introducing a substance that enhances the rate of homologous recombination in the cell, such as for example, by using RAD51 or RAD52.

Embodiments can include replacing at least one allele with a modified allele that includes a combination of amino acids that confer increased resistance to mad cow disease. Embodiments can include replacing a cow gene with a gene from another species that has been genetically modified.

Embodiments of the present invention also relate to a composition including meat from a genetically modified cow, as described more fully herein. The composition, including meat, can be in a packaging material.

Also, in another aspect embodiments of the present invention relate to a method of packaging meat that can include obtaining meat from a genetically modified cow, as described herein, and packaging the meat in a packaging material.

Further, the subject matter disclosed herein relates to a composition, that can include bovine serum or fetal calf serum, from a genetically modified cow as described herein.

Also, one embodiment of the present invention relates to a composition that can include one or more proteins from a genetically modified cow as described herein.

### BRIEF DESCRIPTION OF THE DRAWINGS

FIG. 1 illustrates a “Positive/Negative” homologous recombination construct.

FIG. 2 illustrates a “Gene Trap” homologous recombination construct.

FIG. 3 illustrates an “Over-lapping” homologous recombination construct.

FIG. 4 illustrates the sequence of the Pst I-Bgl II fragment of the HO endonuclease (SEQ ID NO: 4).

FIG. 5 illustrates a sequence for the Fok I endonuclease domain used in chimeric endonucleases (SEQ ID NO: 5).

FIG. 6 illustrates exemplary zinc finger endonuclease strategies.

FIG. 7 illustrates a Sp1C framework for producing a zinc finger protein with three fingers (SEQ ID NO: 8, SEQ ID NO: 9, SEQ ID NO: 10).

FIG. 8 illustrates exemplary primers used to create a zinc finger domain with three fingers (SEQ ID NO: 12, SEQ ID NO: 13, SEQ ID NO: 14, SEQ ID NO: 15).

FIG. 9 illustrates a method of the invention.

FIG. 10 illustrates a method of obtaining a modified animal with a reduced susceptibility to mad cow disease.

FIG. 11 illustrates a scheme for the sequential disruption of both alleles of exon 3 of the PRNP gene.

FIG. 12 illustrates a scheme for the simultaneous disruption of both alleles of exon 3 of the PRNP gene.

FIG. 13 illustrates a vector and method for use in obtaining cow cells in which a gene associated with mad cow disease has been modified.

FIG. 14 illustrates alternative vectors and a method for use in obtaining cow cells in which a gene associated with mad cow disease has been modified.

### DETAILED DESCRIPTION OF THE PREFERRED EMBODIMENT

The present invention relates to cows having a reduced susceptibility to mad cow disease and cells and nucleic acids for generating such cows, as well as methods of obtaining such cows and cells. The present invention also relates to products and compositions derived from cows having a reduced susceptibility to mad cow disease.

One embodiment of the present invention relates to a method of obtaining a cow with reduced susceptibility to mad cow disease. In this embodiment, a cow cell is modified by modifying a gene in the cell that is associated with mad cow disease. The modified gene provides reduced susceptibility to mad cow disease relative to an unmodified gene. Thereafter, a cow is generated from the modified cell.

The cow cell in which the gene associated with mad cow disease is modified can be any cell capable of being used to generate a cow. For example, the cell can be a somatic cell. The somatic cell can be a fibroblast, a granulosa cell and the like. Alternatively, the cell can be a germ cell, a stem cell, and the like. In some embodiments the cell may be any fetal cell.

In some embodiments, the gene associated with mad cow disease can be any gene that contributes to mad cow disease or that aids the spread of variant Creutzfeldt Jacob disease in humans. For example, in some embodiments, the gene which is modified may comprise a gene which encodes an mRNA corresponding to the cDNA nucleotide sequence of SEQ ID NO: 1 (i.e. by “corresponding to the cDNA nucleotide sequence” is meant that the mRNA comprises the sequence of SEQ ID NO: 1 except that the thymidine residues in SEQ ID NO: 1 are uridine residues in the mRNA). Also, the gene may encode a cow prion polypeptide. In some embodiments, the gene may encode a polypeptide comprising the amino acid sequence of SEQ ID NO: 2, which is encoded by nucleotides 162-956 of SEQ ID NO: 1.

In some embodiments the modified cell may be constructed by replacing one or both chromosomal copies of the gene associated with mad cow disease or a portion thereof with a homologous sequence that provides reduced susceptibility to mad cow disease. For example, the homologous sequence may contain a stop codon in the open reading frame which encodes a polypeptide associated with mad cow disease. The stop codon may be introduced into one or both of the chromosomal copies of the gene by homologous recombination. Alternatively, a deletion may be introduced into one or both of the chromosomal copies of the gene by homologous recombination. In some embodiments, the homologous sequence may contain stop codons in all reading frames in the sequence downstream of a deletion to eliminate artifactual translation products. It will be appreciated that a deletion or stop codon may be located at any position which prevents or reduces susceptibility to mad cow disease. Thus, the stop codon or deletion may prevent expression of polypeptides associated with susceptibility to mad cow disease by preventing expression of a fully functional polypeptide in cow cells or via any other mechanism which prevents or reduces susceptibility to mad cow disease.

As discussed above, in some embodiments, the homologous sequence which provides reduced susceptibility to mad cow disease may be a modified form of the cow prion gene, or a portion thereof, which contains a stop codon or deletion. For example, the stop codon or deletion may be constructed in a gene which encodes an mRNA corresponding to the cDNA nucleotide sequence of SEQ ID NO: 1 or a portion of such a gene and may be introduced into one or both chromosomal copies of the gene through homologous recombination. For example, in one embodiment, the homologous sequence can include a genomic DNA region which includes all or a portion of an exon of the cow prion gene which contains a desired modification, such as a stop codon or a deletion. The modified genomic DNA region can then be introduced into one or both chromosomal copies of the gene through homologous recombination.

Alternatively, the modified cell may be constructed by replacing one or more amino acids in one or both chromosomal copies of the gene associated with mad cow disease with an amino acid which provides reduced susceptibility to mad cow disease. For example, the codon encoding the methionine at position 129 of SEQ ID NO: 2 can be replaced with a codon encoding an amino acid other than methionine. In some embodiments, the codon encoding the methionine at position 129 may be replaced by a codon encoding alanine, valine, cysteine, isoleucine, leucine, phenylalanine, tryptophane, or tyrosine. In some embodiments, nucleic acids comprising a gene or portion thereof encoding a polypeptide containing the one or more amino acid replacements are introduced into the chromosome via homologous recombination.

Further, PRNP genes from many if not most animals are very closely related. An allele of the PRNP in sheep has been demonstrated to be resistant to Scrapie (Sheep Mad Cow Disease) personal communication. This resistance is due to a specific combination of amino acids at specific positions within this allele. Therefore, in some embodiments a cow allele or gene can be genetically engineered or modified to include specific combination of amino acids at a specific position in the allele. For example, the cow allele can be modified to include a specific combination of amino acids at the location in the allele or gene that corresponds to the location of the combination in the non cow species.

Alternatively, in one embodiment, the modified cell can be generated by replacing the gene encoding the cow prion protein or a portion thereof with a gene encoding a prion protein from a species other than cow or a portion thereof. Further, the gene from another species can be genetically engineered to reduce susceptibility to mad cow disease, then the gene can replace a gene or allele in a cow. For example, the species other than a cow can include a goat, a mouse, a sheep, a marsupial, and the like.

As discussed above, the modified cells in which one or both chromosomal copies of a gene associated with mad cow disease or a portion thereof has been replaced with a sequence which provides reduced susceptibility to mad cow disease may be constructed using techniques based on homologous recombination. In such methods, a homologous recombination vector is introduced into the cell and homologous recombination is allowed to occur between the chromosomal gene associated with mad cow disease, or a portion thereof, and a homologous sequence which provides reduced susceptibility to mad cow disease such that the chromosomal gene is replaced with a sequence which reduces susceptibility to mad cow disease. In some embodiments, the homologous recombination vector may include a nucleic acid comprising a 5′ region that is homologous to a portion of a gene associated with susceptibility to mad cow disease, a 3′ region that is homologous to a portion of a gene associated with susceptibility to mad cow disease, and at least a portion of the coding sequence of the gene disposed between the 5′ region and the 3′ region. The at least a portion of the coding sequence may include a sequence that reduces susceptibility to mad cow disease.

For example, the at least a portion of the coding sequence may comprise a cow prion gene or a portion thereof which provides reduced susceptibility to mad cow disease. In some embodiments, the at least a portion of the coding sequence may be a modified version of the cow prion gene which contains a stop codon in the open reading frame which encodes the cow prion polypeptide. For example, the at least a portion of the coding region may comprise a modified version of a gene which encodes an mRNA corresponding to the cDNA nucleotide sequence of SEQ ID NO: 1 or a portion of such a gene that contains a stop codon in the open reading frame which encodes the polypeptide of SEQ ID NO: 2. Alternatively, the at least a portion of the coding region may be a modified version of the cow prion gene, such as the cow prion gene which encodes an mRNA corresponding to the cDNA nucleotide sequence of SEQ ID NO: 1, which contains a deletion therein. Replacement of one or both of the chromosomal copies of the gene with the deleted version of the gene confers reduced susceptibility to mad cow disease.

As discussed above, in one embodiment, the homologous sequence can include a genomic DNA region which includes all or a portion of an exon of the cow prion gene which contains a desired modification, such as a stop codon or a deletion. The modified genomic DNA region can then be introduced into one or both chromosomal copies of the gene through homologous recombination.

In some embodiments, the at least a portion of the coding region may comprise a sequence wherein the codon encoding the methionine at position 129 of SEQ ID NO: 2 has been replaced with a codon encoding an amino acid other than methionine. The methionine at position 129 can be replaced, for example, by alanine, valine, cysteine, isoleucine, leucine, phenylalanine, tryptophane, tyrosine, and the like.

In some embodiments, the at least a portion of the coding sequence may comprise a gene encoding a prion protein from a species other than cow or a portion thereof. For example, the species can be a sheep, a goat, a mouse, a marsupial, and the like.

In some embodiments, the homologous recombination vector can further include at least one nucleic acid encoding a detectable polypeptide which may be used to identify modified cells in which homologous recombination has occurred. The nucleic acid that encodes a detectable poplypeptide can be operably linked to a promoter. For example the detectable polypeptide can be CD8, CD4, green fluorescent protein, DsRed2, and the like.

In one embodiment the recombinant nucleic acid can include a nucleic acid encoding CD8 or CD4 operably linked to a promoter and also a nucleic acid encoding green fluorescent protein operably linked to a promoter. In further embodiments, the nucleic acid encoding a detectable polypeptide can be flanked by a site which facilitates recombination. For example, the site that facilitates recombination can be a LoxP site.

In some embodiments of the present invention, the cow cells in which a gene associated with mad cow disease has been modified are then used to generate cows which have a reduced susceptibility to mad cow disease. For example, in some embodiments, the nuclei of the modified cow cells are removed and transferred into enucleated oocytes capable of developing into a genetically modified cow. The oocytes comprising the nuclei from the cow cells are then introduced into a cow in which they can develop into a genetically modified animal. The oocytes are allowed to develop into genetically modified cows and, after birth, the genetically modified cows are raised and bred for any use that is desirable.

For example, the genetically modified cows may be used to provide dairy products, meat products, heterologous proteins introduced through genetic engineering, gelatin, collagen, bovine serum, fetal calf serum, and the like.

It is important for a wide range of medical and veterinary products to develop prion-free sources of bovine and fetal bovine serum. Bovine serum and Fetal Calf Serum (FCS) are used for production of a number of vaccines derived from cells grown in tissue culture, for production of an increasingly long list of highly effective new drugs, the monoclonal antibodies, as well as production of other products injected into humans, such as recombinant proteins. FCS is also essential for much research work that requires growth of cells in culture. At present, each of these activities poses the risk of infection by BSE prions. Replacement of the current Bovine serum and FCS products with prion-free material impacts several large and medically crucial markets with great commercial potential.

In another embodiment the present invention relates to a composition comprising meat from a genetically modified cow having reduced susceptibility to mad cow disease in a packaging material.

Another embodiment of the present invention is a method of packaging meat comprising obtaining meat from a genetically modified cow having reduced susceptibility to mad cow disease and packaging the meat in a packaging material.

Further embodiments of the present invention relate to bovine serum or fetal calf serum from a cow having reduced susceptibility to mad cow disease. Other compositions of the present invention include collagen, gelatin, and the like from a cow having reduced susceptibility to mad cow disease. Another embodiment of the present invention is human proteins produced in a cow having reduced susceptibility to mad cow disease which has also been engineered to produce the human protein.

The following examples are intended to illustrate some embodiments of the present invention. It will be appreciated that the following examples are exemplary only and that the scope of the present invention is defined by the appended claims. Further, it will be appreciated that although certain cells are used in the following examples other cells which are consistent with the intent of the present invention may be substituted.

### EXAMPLE 1

### Generation of Cells in which the Cow Prion Gene has been Modified

Genes associated with susceptibility to mad cow disease are modified in cow cells. In preferred embodiments the cow cells are suitable for use in obtaining genetically modified cows with reduced susceptibility to mad cow disease. For example, the genes may be modified in cells suitable for use in nuclear transfer procedures, stem cell or germ cell-based procedures, and the like. Cells suitable for use in nuclear transfer procedures include but are not limited to one or more of the following cells: primary skin fibroblasts, granulosa cells, and primary fetal fibroblasts, fibroblasts or non-transformed cells from any desired organ or tissue.

Primary cow fibroblasts may be obtained from skin incisions in adult cows. A piece of tissue is removed and placed in tissue-culture media to obtain primary cell lines. (Kubota et al., 2000, Proc. Natl. Acad. Sci. U.S.A. 97(3):990-995, the disclosure of which is incorporated herein by reference in its entirety).

Cow granulosa cells may be obtained as follows (Polejaeva et al., 2000, Nature 407(6800):86-90, the disclosure of which is incorporated herein by reference in its entirety).

Primary cow fetal fibroblasts may be prepared as follows (Schnieke et al., 1997, Science 278(5346):2130-2133, the disclosure of which is incorporated herein by reference in its entirety).

Cells in which one or more genes associated with mad cow disease have been modified may be generated as follows.

Techniques which may be used to modify genes associated with mad cow disease include, but are not limited to the following. In one method, the homologous recombination method described in Capecchi, 1989, Science 244(4910):1288-1292, the disclosure of which is incorporated herein by reference in its entirety, is used to generate modifications. In this method, homologous recombination constructs comprising the coding sequence or a portion of the coding sequence of the gene associated with susceptibility to mad cow disease in which an in frame stop codon has been introduced near the 5′ end of the coding sequence are introduced into the cell using methods such as lipofection, calcium phosphate transfection, electroporation or other methods familiar to those skilled in the art.

It will be appreciated that other methods of modifying genes associated with susceptibility to mad cow disease familiar to those skilled in the art may also be employed. For example, rather than modifying genes associated with susceptibility to mad cow disease by replacing the chromosomal copy of the gene with a gene having a stop codon therein, the chromosomal copy of the gene may be modified by replacing it with a copy of the gene having a deletion in some or all of the coding region. For example, an exon of the gene or a portion thereof may be deleted. In some embodiments, the exon may be in the 5′ region of the gene in order to prevent the expression of a functional protein through alternative splicing. In addition, if desired, the sequence downstream of the deletion may contain stop codons in all reading frames to prevent artifactual translation products.

In another embodiment, the homologous sequence may comprise a prion gene or portion thereof in which the codon encoding the methionine at position 129 of the polypeptide of SEQ ID NO: 2 has been replaced with a codon encoding an amino acid which provides reduced susceptibility to mad cow disease. For example, the codon encoding the methionine at position 129 of SEQ ID NO: 2 may be replaced by a codon encoding alanine, valine, cysteine, isoleucine, leucine, phenylalanine, tryptophane, or tyrosine. In another embodiment, the coding sequence or portion thereof may be a prion gene from an organism other than cow or a portion thereof. For example, in some embodiments, the coding sequence may be a prion gene or portion thereof from a goat, a mouse, a sheep, or a marsupial.

To construct the homologous recombination vector, a nucleic acid comprising the coding sequence of the gene to be modified or a portion thereof is obtained. The nucleic acid may be obtained from a genomic library, a cDNA library, a plasmid or other vector, or any suitable source.

For example, in some embodiments a genomic DNA comprising the coding sequence to be modified or a portion thereof may be obtained as follows. The genomic organization of PrP is known (Horiuchi et al., 1998, Anim. Genet. 29(1):37-40; Lee, I. Y. et al., Genome Res. 8:1022-1037 (1998), the disclosures of which are incorporated herein by reference in its entirety). PCR primers whose entire amplification product lies within a single exon, such as the first exon, the third exon or any other, for example, are designed using GCG's Omiga software. The primers are used to screen a bovine genomic DNA library in a Bacterial Artificial Chromosome (BAC) vector to identify a BAC containing the PrP gene or a portion thereof containing the exon or portion thereof from which the primers are derived. For example, the BAC may be identified by performing a PCR amplification with the above-described primer pair.

Once the BAC is identified and isolated, it is digested into smaller pieces using restriction enzymes. The resultant fragments are separated based upon size by agarose gel electrophoresis. The fragment containing the desired exon of PrP or portion thereof, such as the first exon, is identified and obtained using the same protocol that is used to identify the BAC clone containing the gene, i.e., PCR. This fragment is further characterized using a combination of restriction mapping and DNA sequencing. This provides the raw material from which to make the modified nucleic acid construct as discussed herein.

It will be appreciated that the BAC containing the PrP gene or a desired portion thereof may be identified using any of the methods familiar to those skilled in the art and that the sequence in which the modfication is made may be any sequence which is able to reduce susceptibility to mad cow disease when present in a genetically modified animal. For example, in some embodiments, the modfication is made in a genomic sequence comprising any of the exons, introns, or portions thereof the PrP gene. The modification may be made in any nucleic acid capable of being introduced into a cell which can be used to generate a genetically modified organism. If the modified nucleic acid is to be introduced into the cell via homologous recombination, the genomic DNA is preferably of a size which facilitates homologous recombination.

The nucleic acid comprising the coding sequence of the gene to be modified or a portion thereof may be obtained by excising the desired gene or portion thereof using restriction enzymes or by generating an amplicon comprising the gene or portion thereof by PCR. If desired, the stop codon, deletion, or mutations resulting in one or more amino acid replacements may be introduced into the coding sequence using conventional techniques such as site directed mutagenesis or enzymatic deletion. In some embodiments, the stop codon, deletion, or amino acid replacement(s) is engineered in a gene which encodes an mRNA corresponding to the cDNA nucleotide sequence of SEQ ID NO: 1 or a portion of such a gene.

The disrupted gene or portion thereof, or a gene or portion thereof from a species other than cow, is introduced into a vector suitable for integration into the genome of the cow cells by homologous recombination. Any vector suitable for replacing the chromosomal copies of the gene with the modified gene or gene or portion thereof from a species other than cow may be used. Examples of vector strategies include “Positive/Negative”, “Gene Trap”, “Overlapping” constructs or a construct which inserts a stop codon in all three reading figures. Any of these methods may be used twice if one desires to disrupt both copies of the endogenous target sequence. The main modification is that for the positive/negative, gene trap and over lapping constructs, the second time these constructs are used to knockout a gene, the “positive” marker in each case should be distinguishable from the “positive” marker used in the constructs to knock out the first copy of the gene.

A number of different DNA construct designs can be used to distinguish homologous recombination from random integration, thereby facilitating the identification of cells in which the desired homologous recombination has occurred. Several exemplary DNA constructs used for homologous recombination are provided below. The constructs all provide methods that allow homologous recombination to be efficiently distinguished from random integration.

Positive/Negative Knockout Construct

One type of construct used is a Positive/Negative Knockout Construct. In this construct a “positive” marker is one that indicates that the DNA construct has integrated somewhere in the genome. A “negative” marker is one that indicates that the DNA construct has integrated at random in the genome, (Hanson et al., “Analysis of biological selections for high-efficiency gene targeting,” Mol. Cell Biol. 15 (1):45-51 (1995); the disclosure of which is hereby incorporated by reference in its entirety).

In one embodiment, the “positive” marker is a gene under the control of a constitutively active promoter, for example the promoters of Cyto MegaloVirus (CMV) or the promoter of Simian Virus 40 (SV40). The gene controlled in this way may be an auto-fluorescent protein such as, for example, Enhance Green Fluorescent Protein (EGFP) or DsRed2 (both from Clontech), a gene that encodes resistance to a certain antibiotic (neomycin resistance or hygromycin resistance), a gene encoding a cell surface antigen that can be detected using commercially available antibody, for example CD4 or CD8 (antibodies raised against these proteins come from Rockland, Pharmingen or Jackson), and the like.

In one embodiment, the “negative” marker is also a gene under the control of a constitutively active promoter like that of CMV or SV40. The gene controlled in this way may also be an auto-fluorescent protein such as EGFP or DsRed2 (Clontech), a gene that encodes resistance to a certain antibiotic (neomycin resistance or hygromycin resistance) a gene encoding a cell surface antigen that can be detected by antibodies, and the like. However, the “negative” marker may also be a gene whose product either causes the cell to die by apoptosis, for example, or changes the morphology of the cell in such a way that it is readily detectable by microscopy, for example E-cadherin in early blastocysts.

The “positive” marker is flanked by regions of DNA homologous to genomic DNA. The region lying 5′ to the “positive” marker can be about 1 kB in length, to allow PCR analysis using the primers specific for the “positive” marker and a region of the genome that lies outside of the recombination construct, but may have any length which permits homologous recombination to occur. If the PCR reaction using these primers produces a DNA product of expected size, this is further evidence that a homologous recombination event has occurred. The region to the 3′ of the positive marker can also have any length which permits homologous recombination to occur. Preferably, the 3′ region is as long as possible, but short enough to clone in a bacterial plasmid. For example, the upper range for such a stretch of DNA can be about 10 kB in some embodiments. This 3′ flanking sequence can be at least 3 kB. To the 3′ end of this stretch of genomic DNA the “negative” marker is attached.

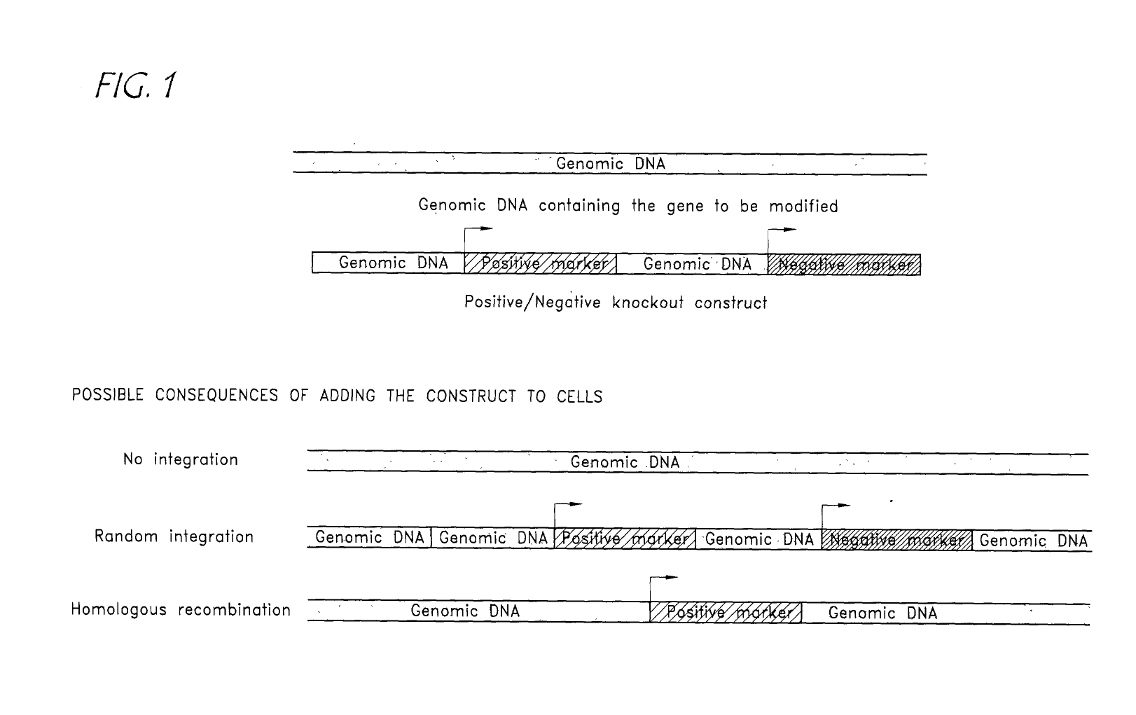

Once this DNA has been introduced into the cell, the cell will fall into one of three phenotypes: (1) No expression of either the “positive” or “negative” marker, for example, where there has been no detectable integration of the DNA construct. (2) Expression of the “positive” and “negative” markers. There may have been a random integration of this construct somewhere within the genome. (3) Expression of the “positive” marker but not the “negative” marker. Homologous recombination may have occurred between the genomic DNA flanking the “positive” marker in the construct and endogenous DNA. In this way the “negative” marker has been lost. These are the desired cells. These three possibilities are shown schematically in FIG. 1.

Gene Trapping Construct

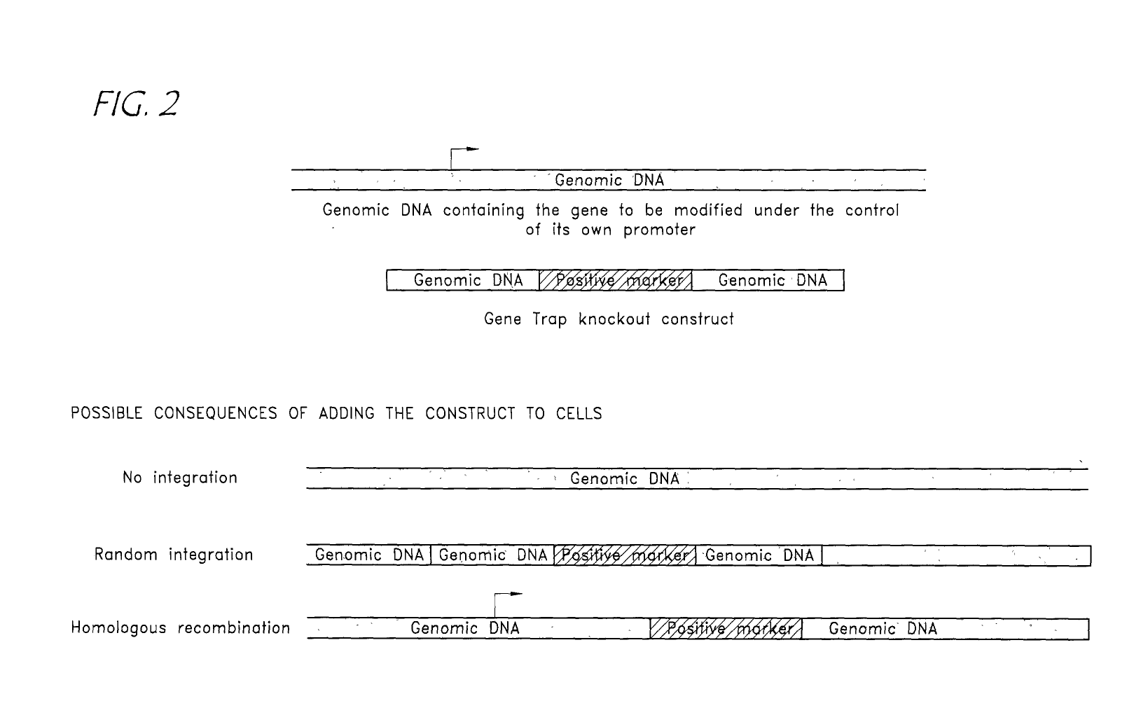

Another type of construct used is called a “Gene Trapping construct.” These constructs contain a promoter-less “positive” marker gene. This gene may be, for example, any of the genes mentioned above for a positive/negative construct. This marker gene is also flanked by pieces of DNA that are homologous to genomic DNA. In this case however, 5′ flanking DNA must put the marker gene under the control of the promoter of the gene to be modified if homologous recombination happens as desired (Sedivy et al., “Positive genetic selection for gene disruption in mammalian cells by homologous recombination,” Proc. Natl. Acad. Sci. U.S.A 86 (1):227-231 (1989); the disclosure of which is hereby incorporated by reference in its entirety). Preferably, this 5′ flanking DNA does not drive expression of the “positive” marker gene by itself. One possible way of doing this is to make a construct where the marker is in frame with the first coding exon of the target gene, but does not include the actual promoter sequences of the gene to be modified. It should be noted that, in preferred embodiments, this technique works if the gene to be modified is expressed at a detectable level in the cell type in which homologous recombination is being attempted. The higher the expression of the endogenous gene the more likely this technique is to work. The region 5′ to the marker can also have any length that permits homologous recombination to occur. Preferably, the 5′ region can be about 1 kB long, to facilitate PCR using primers in the marker and endogenous DNA, in the same way as described above. Similarly, preferably the 3′ flanking region can contain as long a region of homology as possible. An example of an enhancer trapping knockout construct is shown in FIG. 2.

These enhancer trapping based knockout constructs may also contain a 3′ flanking “negative” marker. In this case the DNA construct can be selected for on the basis of three criteria, for example. Expression of the “positive” marker under the control of the endogenous promoter, absence of the “negative” marker, and a positive result of the PCR reaction using the primer pair described above.

Over-Lapping Knockout Construct

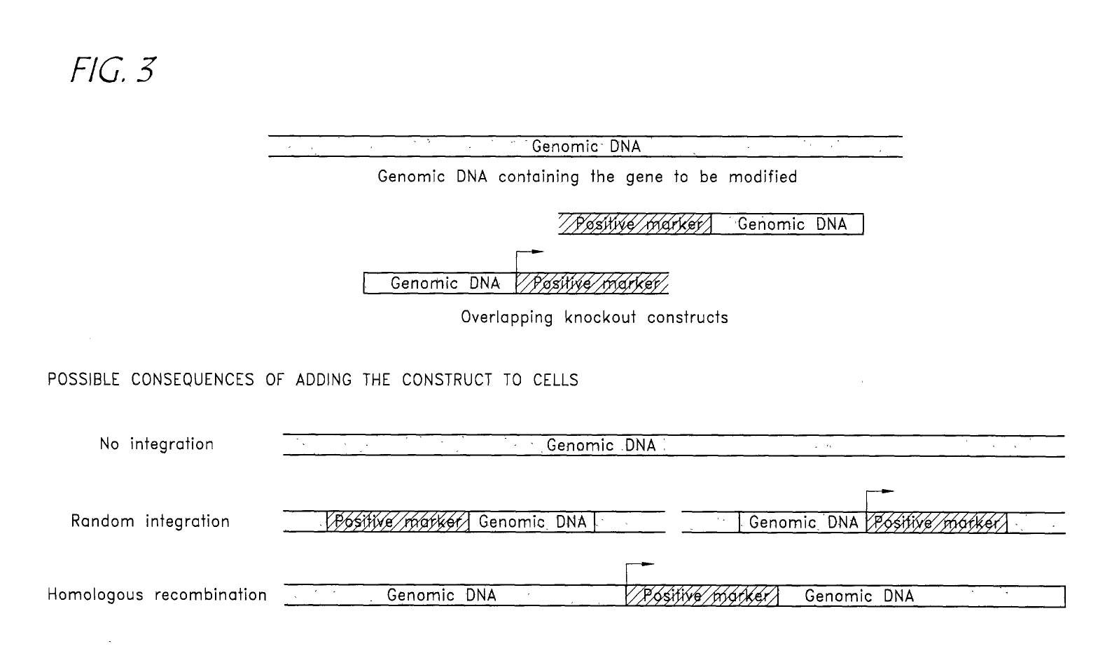

A further type of construct is called an “Over-lapping knockout construct”. This technique uses two DNA constructs (Jallepalli et al., “Securin is required for chromosomal stability in human cells,” Cell 105 (4):445-457 (2001), the disclosure of which is hereby incorporated by reference in its entirety). Each construct contains an overlapping portion of a “positive” marker, but not enough of the marker gene to make a functional reporter protein on its own. The marker is composed of both a constitutively active promoter, for example CMV or SV40 and the coding region for a “positive” marker gene, such as for example, any of those described above. In addition to the marker gene, each of the constructs contains a segment of DNA that flanks the desired integration site. The region of the gene replaced by the “positive” marker is the same size as that marker. If both of these constructs integrate into the genome in such a way as to complete the coding region for the “positive” marker, then that marker is expressed. The chances that both constructs will integrate at random in such an orientation are negligible. Generally, if both constructs integrate by homologous recombination, is it likely that a functional coding region for the “positive” marker will be recreated, and its expression detectable. An example of an overlapping knockout construct is shown in FIG. 3.

Stopper Sequence

Another DNA construct, called a “stopper,” enhances the rate of homologous recombination, but does not contain an intrinsic means of distinguishing homologous recombination from random integration. Unlike the other constructs this one contains no marker genes either “positive” or “negative”. The construct is a stretch of DNA homologous to at least part of the coding region of a gene whose expression is to be removed. The only difference between this piece of DNA and its genomic homolog is that somewhere in region of this DNA that would normally form part of the coding region of the gene, the following sequence, referred to herein as a “stopper” sequence, has been substituted: 5′-ACTAGTTAACTGATCA-3′ (SEQ ID NO: 3). This DNA sequence is 16 bp long, and its introduction adds a stop codon in all three reading frames as well as a recognition site for SpeI and BclI. BclI is methylated by Dam and Dcm methylase activity in bacteria.

Integration by homologous recombination is detectable in two ways. The first method is the most direct, but it requires that the product of the gene being modified is expressed on the surface of the cell, and that there is an antibody that exists that recognizes this protein. If both of these conditions are met, then the introduction of the stop codons truncates the translation of the protein. The truncation shortens the protein so much that it is no longer functional in the cell or detectable by antibodies (either by FACS of Immuno-histochemistry). The second indirect way of checking for integration of the stopper is PCR based. Primers are designed so that one lies outside of the knockout construct, and the other lies within the construct past the position of the stopper. PCR will produce a product whether there has been integration or not. A SpeI restriction digest is carried out on the product of this PCR. If homologous recombination has occurred the stopper will have introduced a novel SpeI site that should be detectable by gel electrophoresis.

Integration of any of the constructs described above by homologous recombination can be verified using a Southern blot. Introduction of the construct will add novel restriction endonuclease sites into the target genomic DNA. If this genomic DNA is digested with appropriate enzymes the DNA flanking the site of recombination is contained in fragments of DNA that are a different size compared to the fragments of genomic DNA which have been digested with the same enzymes but in which homologous recombination has not occurred. Radioactive DNA probes with sequences homologous to these flanking pieces of DNA can be used to detect the change in size of these fragments by Southern blotting using standard methods.

Using either the “Positive/negative”, “Gene Trap” or “Over-lapping” strategies described above, the genetically modified cell ends up with an exogenous marker gene integrated into the genome. In any of these strategies the marker gene and any exogenous regulatory sequences may be flanked by LoxP recombination sites and subsequently removed.

Removal occurs because recombination may occur between two LoxP sites which excises the intervening DNA (Sternberg et al., “Bacteriophage P1 site-specific recombination. II. Recombination between loxP and the bacterial chromosome,” J. Mol. Biol. 150 (4):487-507 (1981); and Sternberg et al., “Bacteriophage P1 site-specific recombination. I. Recombination between loxP sites,” J. Mol. Biol. 150 (4):467-486 (1981); the disclosures of which are both hereby incorporated by reference in their entireties). This recombination is driven by the Cre recombinase (Abremski et al., “Bacteriophage P1 site-specific recombination. Purification and properties of the Cre recombinase protein,” J. Biol. Chem. 259 (3):1509-1514 (1984); the disclosure of which is hereby incorporated by reference in its entirety). This can be provided in cells in which homologous recombination has occurred by introducing it into cells through lipofection (Baubonis et al., “Genomic targeting with purified Cre recombinase,” Nucleic Acids Res. 21 (9):2025-2029 (1993); the disclosure of which is hereby incorporated by reference in its entirety), or by transfecting the cells with a vector comprising an inducible promoter linked to DNA encoding Cre recombinase (Gu et al., “Deletion of a DNA polymerase beta gene segment in T cells using cell type-specific gene targeting,” Science 265 (5168):103-106 (1994); the disclosure of which is hereby incorporated by reference in its entirety).

It will be appreciated that rather than using a recombination vector comprising a disruption in the coding sequence of the gene associated with mad cow disease, the recombination vector may contain a sequence which introduces a deletion in the target gene or a sequence which disrupts the gene in some other manner, such as by disrupting the promoter from which transcription of the target gene initiates.

If both functional copies of the gene associated with mad cow disease have been disrupted, then the stopper sequence described above has worked. It will also be appreciated that the “Positive/Negative”, “Gene Trap” and “Overlapping constructs” described above may be used twice if one desires to disrupt both copies of the endogenous target sequence. The main modification is that the second time these constructs are used to knockout a gene associated with mad cow disease, the “positive” marker in each case should be distinguishable from the “positive” marker used in the constructs to knock out the first copy of the gene.

Additionally, if desired, the cells in which the gene modifications are to be generated may be transfected with a nucleic acid which encodes telomerase such that the transfected cell expresses telomerase. Telomeres are important because they protect chromosomes from degradation and fusion. (Dymecki, S. M., “Flp recombinase promotes site-specific DNA recombination in embryonic stem cells and transgenic mice,” Proc. Natl. Acad. Sci. USA, 93:6191-6 (1996)). Normally during cell division, the telomeres are gradually consumed. This results in, after a certain number of divisions, the cells aging or going into senescence; they do not divide anymore. Expression of telomerase may reduce or prevent senescence in the cells in which the modifications are to be generated. Telomerases protect chromosomes from degradation and fusion. Normally during cell division the telomeres at the ends of the chromosomes are gradually consumed and after a certain number of divisions, the cells age or go into senescence. Over-expression of telomerase has been shown to allow cells to continue to divide without becoming transformed into malignant cells (Allsopp et al., 1995, Exp. Cell Res. 220:194-200, the disclosure of which is incorporated herein by reference in its entirety). Telomerase may be expressed in the cells using any of the methods familiar to those skilled in the art. For example, if desired the expression of telomerase may be regulated by flanking the telomerase gene with FRT sites (the target for Flp recombinase) such that the telomerase gene can be deleted once cells having all of the desired genes disrupted have been generated (See Dymecki, 1996, PNAS 93:6191-6196, the disclosure of which is incorporated herein by reference in its entirety.

If desired, rather than modifying both chromosomal copies of a gene associated with mad cow disease in a single cell, a single copy of the gene may be modified in cell lines obtained from primary cells and nuclear transfer may be performed as described below to make a cow in which a single chromosomal copy of the gene associated with mad cow disease has been modified. Cells carrying the modified gene are harvested from the cow as early as possible to obtain cell lines for use in generating a cow in which the second chromosomal copy of the gene associated with mad cow disease has been modified. For example, the cells for generating the next modification may be obtained at the early embryonic stage. Alternatively, the cells for generating the next modification may be obtained from older animals.

It will be appreciated that other methodologies for generating cells and cows in which genes associated with susceptibility to mad cow disease have been modified may also be employed. For example, stem-cell or germ cell based methods may be used to obtain cows in which a gene associated with susceptibility to mad cow disease has been modified. In such methods, stem cells or germ cells are obtained from the cow. For example, the stem cells or germ cells may be obtained as described in U.S. Pat. No. 6,194,635, the disclosure of which is herein incorporated by reference in its entirety. A homologous recombination vector comprising a selectable marker, for example a gene conferring resistance to the drug G418, and the modified gene or portion thereof, or a gene or portion thereof from a species other than cow, which is to replace the chromosomal gene is introduced into the stem cells. Cells expressing the selectable marker are identified and their chromosomal DNA is analyzed as described above to determine whether the modified gene or portion thereof or gene or portion thereof from a species other than cow has integrated into the cellular genome via a homologous recombination event or whether the modified gene or portion thereof or gene or portion thereof from a species other than cow has integrated randomly. Cells in which homologous recombination has occurred are injected into a fertilized embryo and implanted into a surrogate mother as described in U.S. Pat. No. 6,194,635, the disclosure of which is herein incorporated by reference in its entirety. The resulting offspring are chimeric and are bred to generate heterozygous animals. The heterozygous animals are then bred to generate animals homozygous for the modified gene.

Enhancing Homologous Recombination

If desired, the frequency of homologous recombination may be enhanced using a variety of methods familiar to those skilled in the art. For example, the RecA system described in Kowalczykowski et al., 1994, Microbiol. Rev. 58(3):401-465, the disclosure of which is incorporated herein by reference in its entirety may be used to enhance the frequency of homologous recombination events. Briefly, in this procedure the homologous recombination vector comprising the modified gene is contacted with RecA under conditions which permit RecA to bind to the sequence to be incorporated into the genome of the host organism. The sequence of the modified gene, which is coated with RecA, is then introduced into the cell in which the gene is to be modified as described above.

Also, homologous recombination can be enhanced by introducing or expressing factors that enhance the rate of homologous recombination throughout the genome. For example, the RAD51 system is one such enhancement. (Yanew and Porter, Gene Ther. 6:1282-90 (1999), which is hereby incorporated by reference in its entirety). RAD51 promotes the binding and insertion of a DNA strand into the homologous sequence of the endogenous DNA.

Another method of specifically altering genomic DNA containing target genes at a higher rate than using traditional homologous recombination is the use of recombinagenic oligonucleotides or “Genoplasts™” to insert point mutations into intact chromosomes. Self-complementary chimeric oligonucleotides that consist of DNA and 2′-O-methyl RNA nucleotides arranged into a double-hairpin configuration can elicit a point mutation when targeted to a gene sequence. (Gamper H. B. et al., “A plausible mechanism for gene correction by chimeric oligonucleotides,” Biochemistry 39:5808-16 (2000), which is hereby incorporated by reference in its entirety). Such a point mutation could change an early codon in a gene of interest into a “stop” codon, thus preventing translation of that gene.

Alternatively or in combination with the above, the frequency of homologous recombination may be enhanced using double stranded breaks in the genomic region where it is desired for homologous recombination to occur. The present invention provides more efficient methods for generating genetically modified cells which can be used to obtain genetically modified organisms. In some embodiments of the present invention, a cell capable of generating a desired organism is obtained. Preferably the cell is a primary cell. The cell contains an endogenous nucleotide sequence at or near which it is desired to have homologous recombination occur in order to generate an organism containing a desired genetic modification. The frequency of homologous recombination at or near the endogenous nucleotide sequence is enhanced by cleaving the endogenous nucleotide sequence in the cell with an endonuclease. Preferably, both strands of the endogenous nucleotide sequence are cleaved by the endonuclease. A nucleic acid comprising a nucleotide sequence homologous to at least a portion of the chromosomal region containing or adjacent to the endogenous nucleotide sequence at which the endonuclease cleaves is introduced into the cell such that homologous recombination occurs between the nucleic acid and the chromosomal target sequence. Thereafter, a cell in which the desired homologous recombination event has occurred may be identified and used to generate a genetically modified organism using techniques such as nuclear transfer.

In some embodiments, the frequency of homologous recombination is enhanced using the method described in Cohen-Tannoudji et al., 1998, Mol. Cell. Biol. 18(3):1444-1448, the disclosure of which is incorporated herein by reference in its entirety. Briefly, this strategy induces an endogenous gap repair process at a defined location within the genome by induction of a double-stranded break in the gene to be disrupted. In turn, the double-stranded break increases the frequency of recombination. Double-stranded breaks are introduced into the chromosomal target genes by introducing an I-SceI yeast meganuclease restriction site into the chromosomal target genes in the cells. Thereafter, I-SceI yeast meganuclease is introduced into the cells using a transient expression vector and the homologous recombination vector bearing the disrupted target gene is also introduced into the cells.

In preferred embodiments of the present invention, zinc finger endonucleases (ZFEs) are used to enhance the rate of homologous recombination in the cow cells. The cells may be any type of cell which is capable of being used to generate a genetically modified organism or tissue. For example, in some embodiments, the cell may be primary skin fibroblasts, granulosa cells, primary fetal fibroblasts, stem cells, germ cells, fibroblasts or non-transformed cells from any desired organ or tissue.

In some embodiments of the present invention, a ZFE is used to cleave an endogenous chromosomal nucleotide sequence at or near which it is desired to introduce a nucleic acid by homologous recombination. The ZFE comprises a zinc finger domain which binds near the endogenous nucleotide sequence at which is to be cleaved and an endonuclease domain which cleaves the endogenous chromosomal nucleotide sequence. As mentioned, above, cleavage of the endogenous chromosomal nucleotide sequence increases the frequency of homologous recombination at or near that nucleotide sequence. In some embodiments, the ZFEs can also include a purification tag which facilitates the purification of the ZFE.

Any suitable endonuclease domain can be used to cleave the endogenous chromosomal nucleotide sequence. The endonuclease domain is fused to the heterologous DNA binding domain (such as a zinc finger DNA binding domain) such that the endonuclease will cleave the endogenous chromosomal DNA at the desired nucleotide sequence. As discussed below, in some embodiments the endonuclease domain can be the HO endonuclease. In more preferred embodiments the endonuclease domain may be from the Fok I endonuclease. One of skill in the art will appreciate that any other endonuclease domain that is capable of working with heterologous DNA binding domains, preferably with zinc finger DNA binding domains, can be used.

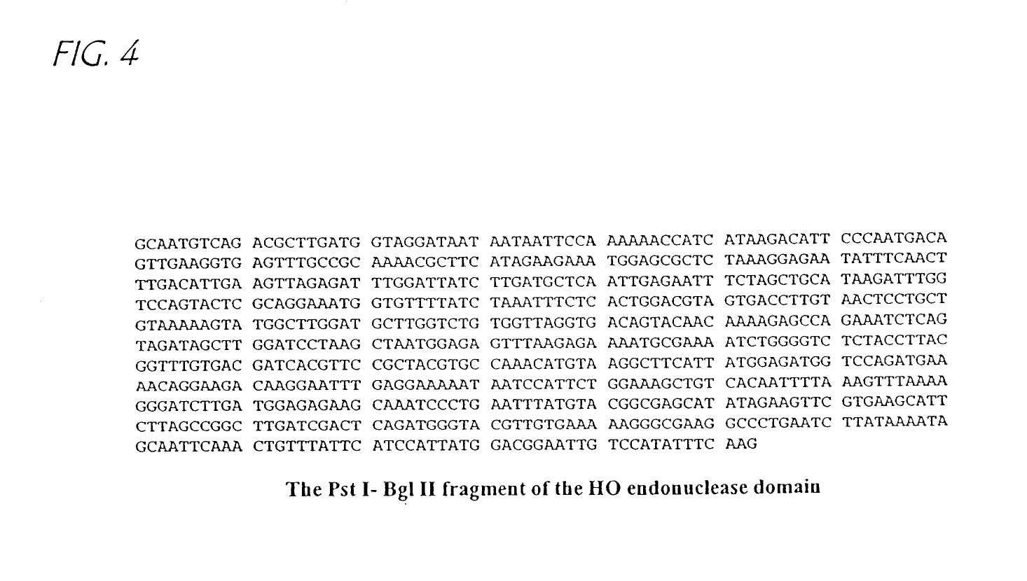

The HO endonuclease domain from Saccharomyces cerevisiae is encoded by a 753 bp Pst I-Bgl II fragment of the HO endonuclease cDNA available on Pubmed (Acc # X90957, the disclosure of which is incorporated herein by reference in its entirety). The HO endonuclease cuts both strands of DNA (Nahon et al., “Targeting a truncated Ho-endonuclease of yeast to novel DNA sites with foreign zinc fingers,” Nucleic Acids Res. 26 (5):1233-1239 (1998); the disclosure of which is incorporated herein by reference in its entirety). FIG. 4 illustrates the sequence of the Pst I-Bgl II fragment of the HO endonuclease cDNA (SEQ ID NO: 4) which may be used in the ZFEs of the present invention. Saccharomyces cerevisiae genes rarely contain any introns, so, if desired, the HO gene can be cloned directly from genomic DNA prepared by standard methods. For example, if desired, the HO endonuclease domain can be cloned using standard PCR methods.

In some embodiments, the Fok I (Flavobacterium okeanokoites) endonuclease may be fused to a heterologous DNA binding domain. The Fok I endonuclease domain functions independently of the DNA binding domain and cuts a double stranded DNA only as a dimer (the monomer does not cut DNA) (Li et al., “Functional domains in Fok I restriction endonuclease,” Proc. Natl. Acad. Sci. U.S.A 89 (10):4275-4279 (1992), and Kim et al., “Hybrid restriction enzymes: zinc finger fusions to Fok I cleavage domain,” Proc. Natl. Acad. Sci. U.S.A 93 (3):1156-1160 (1996); the disclosures of which are incorporated herein by reference in their entireties). Therefore, in order to create double stranded DNA breaks, two ZFEs are positioned so that the Fok I domains they contain dimerise.

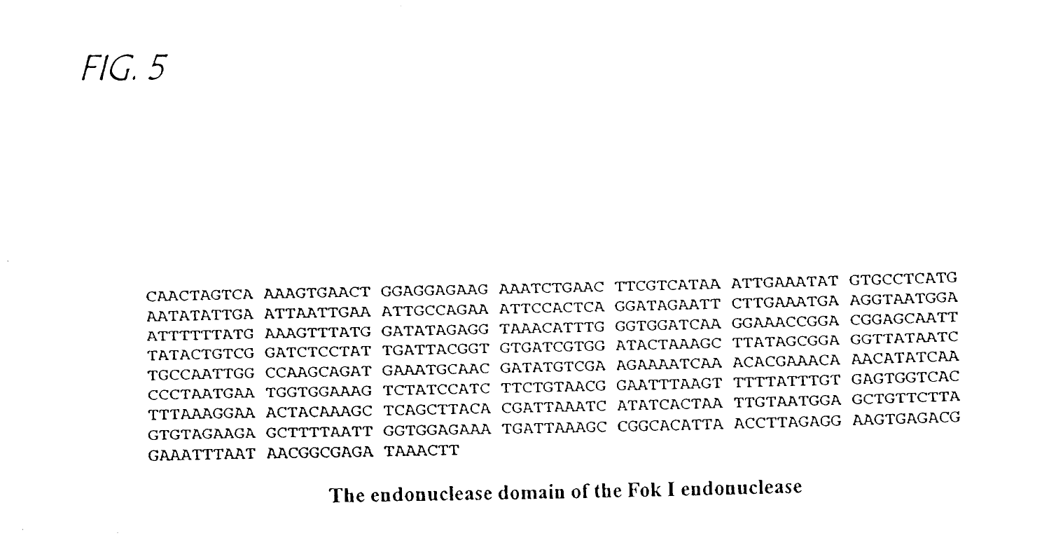

The Fok I endonuclease domain can be cloned by PCR from the genomic DNA of the marine bacteria Flavobacterium okeanokoites (ATCC) prepared by standard methods. The sequence of the Fok I endonuclease is available on Pubmed (Acc # M28828 and Acc # J04623, the disclosures of which are incorporated herein by reference in their entireties). FIG. 5 depicts the sequence of the Fok I endonuclease domain (SEQ ID NO: 5) that can be used in chimeric endonucleases such as those utilized in the present methods.

Again, it will be appreciated that any other endonuclease domain that works with heterologous DNA binding domains can be fused to the zinc finger DNA binding domain.

As mentioned above, the ZFE includes a zinc finger domain with specific binding affinity for a desired specific target sequence. In preferred embodiments, the ZFE specifically binds to an endogenous chromosomal DNA sequence. The specific nucleic acid sequence or more preferably specific endogenous chromosomal sequence can be any sequence in a nucleic acid region where it is desired to enhance homologous recombination. For example, the nucleic acid region may be a region which contains a gene in which it is desired to introduce a mutation, such as a point mutation or deletion, or a region into which it is desired to introduce a gene conferring a desired phenotype.

There are a large number of naturally occurring zinc finger DNA binding proteins which contain zinc finger domains that may be incorporated into a ZFE designed to bind to a specific endogenous chromosomal sequence. Each individual “zinc finger” in the ZFE recognizes a stretch of three consecutive nucleic acid base pairs. The ZFE may have a variable number of zinc fingers. For example, ZFEs with between one and six zinc fingers can be designed. In other examples, more than six fingers can be used. A two finger protein has a recognition sequence of six base pairs, a three finger protein has a recognition sequence of nine base pairs and so on. Therefore, the ZFEs used in the methods of the present invention may be designed to recognize any desired endogenous chromosomal target sequence, thereby avoiding the necessity of introducing a cleavage site recognized by the endonuclease into the genome through genetic engineering.

In preferred embodiments the ZFE protein can be designed and/or constructed to recognize a site which is present only once in the genome of a cell. For example, one ZFE protein can be designed and made with at least five zinc fingers. Also, more than one ZFE protein can be designed and made so that collectively the ZFEs have five zinc fingers (i.e. a ZFE having two zinc fingers may complex with a ZFE having 3 zinc fingers to yield a complex with five zinc fingers). Five is used here only as an exemplary number. Any other number of fingers can be used. Five zinc fingers, either individually or in combination, have a recognition sequence of at least fifteen base pairs. Statistically, a ZFE with 5 fingers will cut the genome once every 4¹⁵ (about 1×10⁹) base pairs, which should be less than once per average size genome. In more preferred embodiments, an individual protein or a combination of proteins with six zinc fingers can be used. Such proteins have a recognition sequence of 18 bp.

Appropriate ZFE domains can be designed based upon many different considerations. For example, use of a particular endonuclease may contribute to design considerations for a particular ZFE. As an exemplary illustration, the yeast HO domain can be linked to a single protein that contains six zinc fingers because the HO domain cuts both strands of DNA. Further discussion of the design of sequence specific ZFEs is presented below.

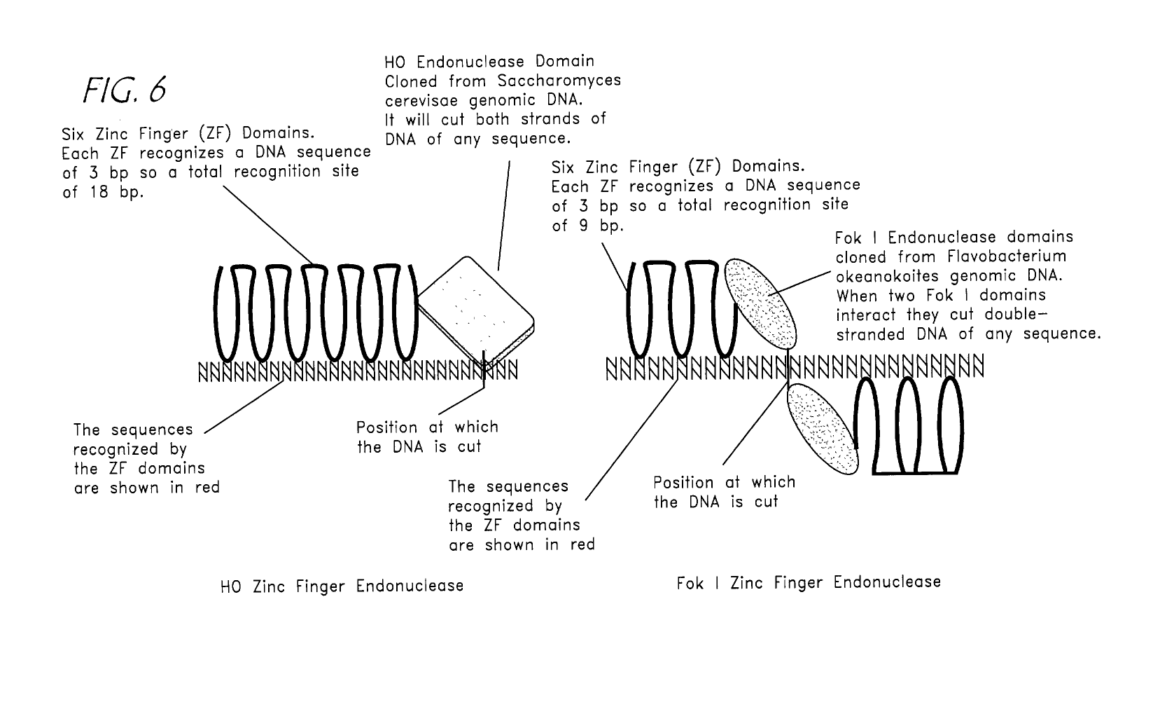

Alternatively, the Fok I endonuclease domain only cuts double stranded DNA as a dimer. Therefore, two ZFE proteins can be made and used in the methods of the present invention. These ZFEs can each have a Fok I endonuclease domain and a zinc finger domain with three fingers. They can be designed so that both Fok I ZFEs bind to the DNA and dimerise. In such cases, these two ZFEs in combination have a recognition site of 18 bp and cut both strands of DNA. FIG. 6 illustrates examples of a ZFE that includes an HO endonuclease, and ZFEs using the Fok I endonuclease. Each ZFE in FIG. 6 has an 18 bp recognition site and cuts both strands of double stranded DNA.

The particular zinc fingers used in the ZFE will depend on the target sequence of interest. A target sequence in which it is desired to increase the frequency of homologous recombination can be scanned to identify binding sites therein which will be recognized by the zinc finger domain of a ZFE. The scanning can be accomplished either manually (for example, by eye) or using DNA analysis software, such as MacVector (Macintosh) or Omiga 2.0 (PC), both produced by the Genetics Computer Group. For a pair of Fok I containing ZFEs, two zinc finger proteins, each with three fingers, bind DNA in a mirror image orientation, with a space of 6 bp in between the two. For example, the sequence that is scanned for can be 5′-G/A N N G/A N N G/A N N N N N N N N N N C/T N N C/T N N C/T-3′ (SEQ ID NO: 6). If a six finger protein with an HO endonuclease domain attached is used, then the desired target sequence can be 5′-G/A N N G/A N N G/A N N G/A N N G/A N N G/A N N-3′ (SEQ ID NO: 7), for example. In these examples, if “N” is any base pair, then all of the zinc fingers that bind to any sequence “GNN” and “ANN” are already determined (Segal et al., “Toward controlling gene expression at will: selection and design of zinc finger domains recognizing each of the 5′-GNN-3′ DNA target sequences,” Proc. Natl. Acad. Sci. U.S.A 96 (6):2758-2763 (1999), and Dreier et al., “Development of zinc finger domains for recognition of the 5′-ANN-3′ family of DNA sequences and their use in the construction of artificial transcription factors,” J. Biol. Chem. 276 (31):29466-29478 (2001); the disclosure of which are incorporated herein by reference in their entireties).

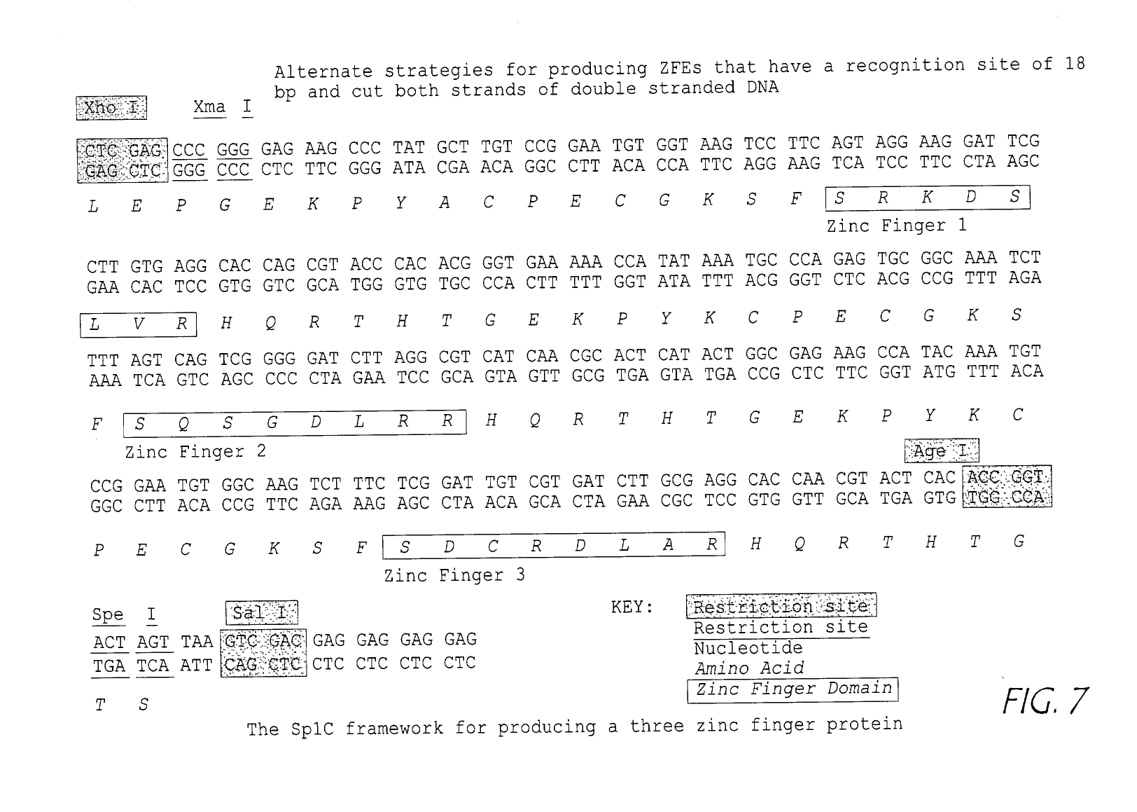

The sequence encoding the identified zinc fingers can be cloned into a vector according well known methods in the art. In one example, FIG. 7 (SEQ ID NO: 8, SEQ ID NO: 9, SEQ ID NO: 10) illustrates one possible peptide framework into which any three zinc fingers that recognize consecutive base pair triplets can be cloned. Any individual zinc finger coding region can be substituted at the positions marked for zinc finger 1, zinc finger 2 and zinc finger 3. In this particular example zinc finger 1 recognizes “GTG”, zinc finger 2 “GCA” and zinc finger 3 “GCC”, so all together this protein will recognize “GTGGCAGCC”. Restriction sites are present on either side of this sequence to facilitate cloning. The backbone peptide in this case is that of Sp1C, a consensus sequence framework based on the human transcription factor Sp1 (Desjarlais et al., “Use of a zinc-finger consensus sequence framework and specificity rules to design specific DNA binding proteins,” Proc. Natl. Acad. Sci. U.S.A 90 (6):2256-2260 (1993); the disclosure of which is incorporated herein by reference in its entirety).

Sp1C is a three finger network and as such can be the zinc finger DNA binding domain that is linked to the Fok I endonuclease domain. Using the restriction sites Age I and Xma I two three-finger coding regions can be joined to form a six-finger protein with the same consensus linker (TGEKP) (SEQ ID NO: 11) between all fingers. This technique is described in (Desjarlais et al., “Use of a zinc-finger consensus sequence framework and specificity rules to design specific DNA binding proteins,” Proc. Natl. Acad. Sci. U.S.A 90 (6):2256-2260 (1993); the disclosure of which is incorporated herein by reference in its entirety.) This six finger framework can be the zinc finger DNA binding domain that is linked to a desired endonuclease domain. The skilled artisan will appreciate that many other frameworks can be used to clone sequences encoding a plurality of zinc fingers.

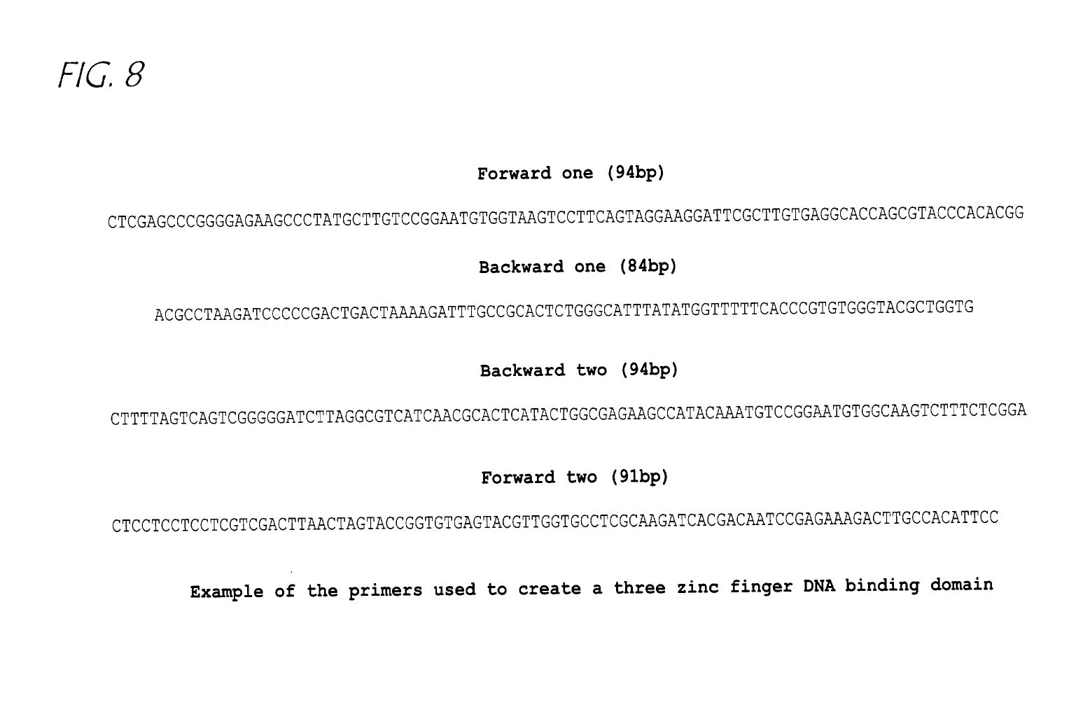

The sequence in FIG. 7 can be constructed using standard PCR methods. FIG. 8 illustrates exemplary PCR primers that can be used. Two 94 bp “forward” primers (SEQ ID NO: 12, SEQ ID NO: 14) can encode the 5′ strand, and two “backward” primers that overlap these “forward” primers, one 84 bp (SEQ ID NO: 13) the other 91 bp (SEQ ID NO: 15), can encode the 3′ strand. These primers can provide both the primers and the template when mixed together in a PCR reaction.

It will be appreciated that the zinc fingers in the ZFEs used in the methods of the present invention may be any combination of zinc fingers which recognize the desired binding site. The zinc fingers may come from the same protein or from any combination of heterologous proteins which yields the desired binding site.

A nucleotide sequence encoding a ZFE with the desired number of fingers fused to the desired endonuclease is cloned into a desired expression vector. There are a number of commercially available expression vectors into which the nucleotide sequence encoding the ZFE can be cloned. The expression vector is then introduced into a cell capable of producing an active ZFE. For example, the expression vector may be introduced into a bacterial cell, a yeast cell, an insect cell or a mammalian cell. Preferably, the cell lacks the binding site recognized by the ZFE. Alternatively, the cell may contain the binding site recognized by the ZFE but the site may be protected from cleavage by the endonuclease through the action of cellular enzymes.

In other embodiments, the ZFE can be expressed or produced in a cell free system such as TNT Reticulocyte Lysate. The produced ZFE can be purified by any appropriate method, including those discussed more fully herein. In preferred embodiments, the ZFE also includes a purification tag which facilitates purification of the ZFE. For example, the purification tag may be the maltose binding protein, myc epitope, a poly-histidine tag, HA tag, FLAG-tag, GST-tag, or other tags familiar to those skilled in the art. In other embodiments, the purification tag may be a peptide which is recognized by an antibody which may be linked to a solid support such as a chromatography column.

Many commercially available expression systems include purification tags, which can be used with the embodiments of the invention. Three examples of this are pET-14b (Novagen) which produces a Histidine tagged protein produced under the control of T7 polymerase. This vector is suitable for use with TNT Reticulocyte Lysate (Promega). The pMal system (New England Biolabs) which produces maltose binding protein tagged proteins under the control of the malE promoter in bacteria may also be used. The pcDNA vectors (Invitrogen) which produce proteins tagged with many different purification tags in a way that is suitable for expression in mammalian cells may also be used.

The ZFE produced as described above is purified using conventional techniques such as a chromatography column containing moieties thereon which bind to the purification tag. The purified ZFE is then quantified and the desired amount of ZFE is introduced into the cells in which it is desired to enhance the frequency of homologous recombination. The ZFE may be introduced into the cells using any desired technique. In a preferred embodiment, the ZFE is microinjected into the cells.

Alternatively, rather than purifying the ZFE and introducing it into the cells in which it is desired to enhance the frequency of homologous recombination, the ZFE may be expressed directly in the cells. In such embodiments, an expression vector containing a nucleotide sequence encoding the ZFE operably linked to a promoter is introduced into the cells. The promoter may be a constitutive promoter or a regulated promoter. The expression vector may be a transient expression vector or a vector which integrates into the genome of the cells.

A recombination vector comprising a 5′ region homologous to at least a portion of the chromosomal region in which homologous recombination is desired and a 3′ region homologous to at least a portion of the chromosomal region in which homologous recombination is introduced into the cell. The lengths of the 5′ region and the 3′ region may be any lengths which permit homologous recombination to occur. The recombination also contains an insertion sequence located between the 5′ region and the 3′ region. The insertion sequence is a sequence which is desired to be introduced into the genome of the cell. Introduction of the insertion sequence into the genome of the cell disrupts a gene associated with mad cow disease.

In some embodiments, the insertion sequence introduces a point mutation into the target gene associated with mad cow disease after homologous recombination has occurred. The point mutation disrupts the endogenous chromosomal gene. In other embodiments, the insertion sequence introduces a deletion into the gene associated with mad cow disease after homologous recombination has occurred. In such embodiments, the insertion sequence may “knock out” the target gene.

In some embodiments, it may be desired to disrupt or knock-out both chromosomal copies of the target gene associated with mad cow disease. In such embodiments, two homologous recombination procedures are performed as described herein to disrupt both copies of the chromosomal target sequence. Alternatively, a genetically modified organism in which one copy of the chromosomal target sequence has been disrupted desired may be generated using the methods described herein. Subsequently, cells may be obtained from the genetically modified organism and subjected to a second homologous recombination procedure as described herein. The cells from the second homologous recombination procedure may then be used to generate an organism in which both chromosomal copies of the target sequence have been disrupted as desired.

In some embodiments, the insertion sequence or a portion thereof may be located between two sites, such as loxP sites, which allow the insertion sequence or a portion thereof to be deleted from the genome of the cell at a desired time. In embodiments in which the insertion sequence or a portion thereof is located between loxP sites, the insertion sequence or portion thereof may be removed from the genome of the cell by providing the Cre protein. Cre may be provided in the cells in which a homologous recombination event has occurred by introducing Cre into the cells through lipofection (Baubonis et al., 1993, Nucleic Acids Res. 21:2025-9, the disclosure of which is incorporated herein by reference in its entirety), or by transfecting the cells with a vector comprising an inducible promoter operably linked to a nucleic acid encoding Cre (Gu et al., 1994, Science 265:103-106; the disclosure of which is incorporated herein by reference in its entirety).

In some embodiments, the recombination vector comprises a nucleotide sequence which encodes a detectable or selectable marker which facilitates the identification or selection of cells in which the desired homologous recombination event has occurred. For example, in some embodiments the recombination vector may comprise a selectable marker which provides resistance to a drug.

In other preferred embodiments the detectable marker may be a cell surface protein which is recognized by an antibody such that cells expressing the cell surface marker may be isolated using FACS. For example, if the somatic cells are subject to too harsh treatment during genetic modification in vitro and storage, the nuclear transfer is very inefficient and it may also affect the health of the cloned animal. Primary cells enter a stage of senescence after a number of divisions, i.e., they stop dividing which then will not allow enough time for the genetic modifications. In addition, it is generally unhealthy for the cells to be in culture for prolonged periods of time. Common problems with cloned animals are organ failures such as hydroallantois, distension of the liver, heart and liver insufficiencies. (McCreath, K. J. et al., “Production of gene-targeted sheep by nuclear transfer from cultured somatic cells,” Nature (2000)). In some cases screening for cells with genetically modified genes (transgenic or knock-out) can be time consuming, and also toxic for the cells. Screening by FACS and/or by magnetic beads can avoid such time consumption and toxicity. In addition, the methods of the present invention are also fast which will allow for more extensive genetic manipulations of the cells.

The recombination vector may be introduced into the cell concurrently with the ZFE, prior to the ZFE, or after the ZFE. Cleavage of the chromosomal DNA by the ZFE enhances the frequency of homologous recombination by the recombination vector. Cells in which the desired recombination event has occurred are identified and, if desired, the chromosomal structure of the cells may verified using techniques such as PCR or Southern blotting. Further discussion of recombination vectors and methods for their use is provided in Examples 1G and 1H, and several exemplary constructs are provided above in relation to FIGS. 1-3.

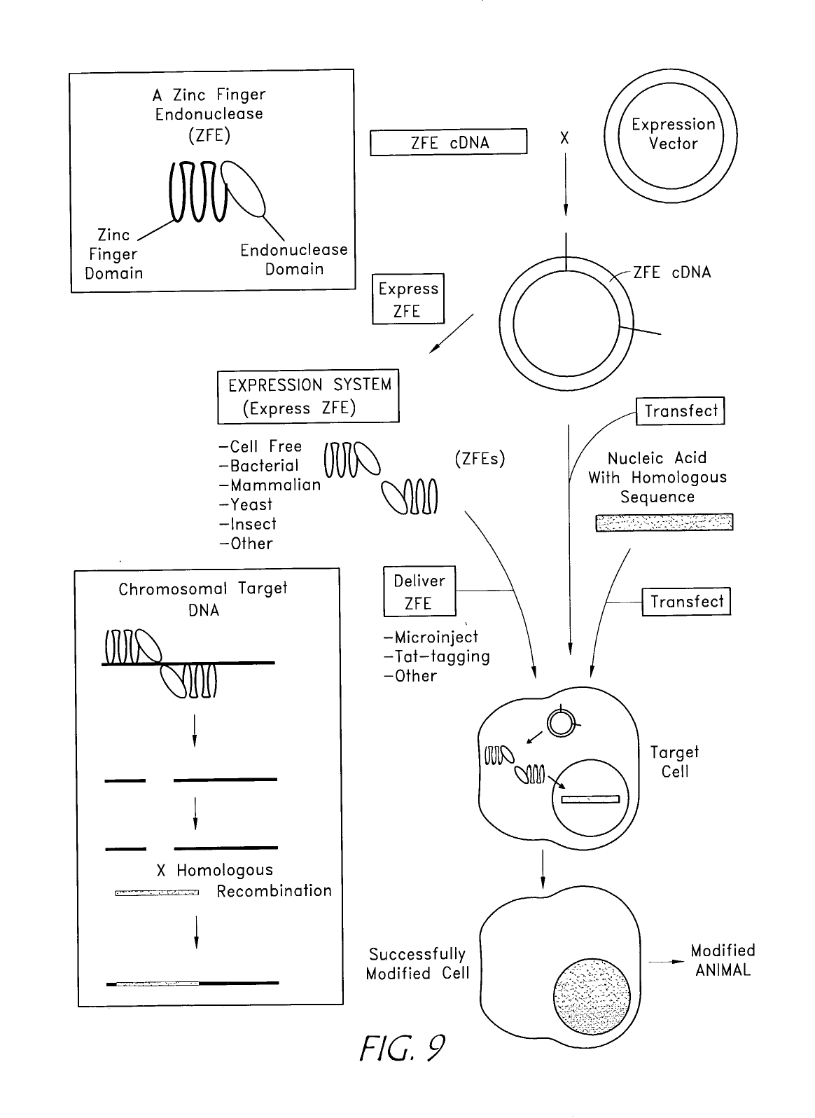

FIG. 9 illustrates a method of the present invention.

### EXAMPLE 1A

### Design of a Zinc Finger Endonuclease

A ZFE is designed with an endonuclease domain that cuts DNA and a zinc finger domain which recognizes the specific DNA sequence “GTGGCAGCC.” The zinc finger domains encoded by the sequence illustrated in FIG. 7 are fused to the Fok I endonuclease.

A standard PCR protocol is performed using the primers illustrated in FIG. 8 in order to make and amplify the zinc finger domain encoded by the sequence in FIG. 7. The Fok I sequence illustrated in FIG. 5 is amplified using standard PCR methods. The amplified zinc finger domain sequence is joined to the amplified Fok I construct thereby forming a chimeric DNA sequence.

### EXAMPLE 1B

### Design of 6-mer Endonuclease Domain

The zinc finger coding domains of FIG. 7 are cut using the restriction sites Age I and Xma I. The two three-finger coding domains are joined to form a six-finger coding domain with the same consensus linker (TGEKP) (SEQ ID NO: 11) between all fingers. This six finger framework is linked to the HO endonuclease domain illustrated in FIG. 4.

### EXAMPLE 1C

### Design of a Sequence Specific ZFE

A target endogenous chromosomal nucleotide sequence at or near which it is desired to enhance the frequency of homologous recombination is identified and scanned to identify a sequence which will be bound by a zinc finger protein comprising 6 zinc finger domains. If “N” is any base pair, then the zinc fingers are selected to bind to the following sequence within the target nucleic acid: 5′-G/A N N G/A N N G/A N N G/A N N G/A N N G/A N N-3′ (SEQ ID NO: 7), where N is A, G, C or T.

### EXAMPLE 1D

### Design of a Sequence Specific ZFE

A target endogenous chromosomal target sequence at or near which it is desired to enhance the frequency of homologous recombination is identified and scanned to identify a nucleotide sequence which will be recognized by a ZFE. Two 3-mer zinc finger domains for use with the Fok I endonuclease are designed by determining a zinc finger protein that will specifically bind to the target DNA in a mirror image orientation, with a space of 6 bp in between the two. If “N” is A, G, C or T, then all of the zinc fingers that bind to any sequence “GNN” and “ANN” are known. The zinc finger domain is selected to bind to the sequence 5′-G/A N N G/A N N G/A N N N N N N N N N N C/T N N C/T N N C/T-3′ (SEQ ID NO: 6).

### EXAMPLE 1E

### Expression of the ZFE

The construct of Example 1A or 1B is introduced into the pMal bacterial expression vector (New England Biolabs) and expressed. The ZFE protein is expressed under the control of the malE promoter in bacteria tagged with a maltose binding protein. The ZFE protein is purified by maltose chromatography and quantified.

### EXAMPLE 1F

### Generation of a Cow Cell in which Both Chromosomal Copies of a Target Gene are Disrupted

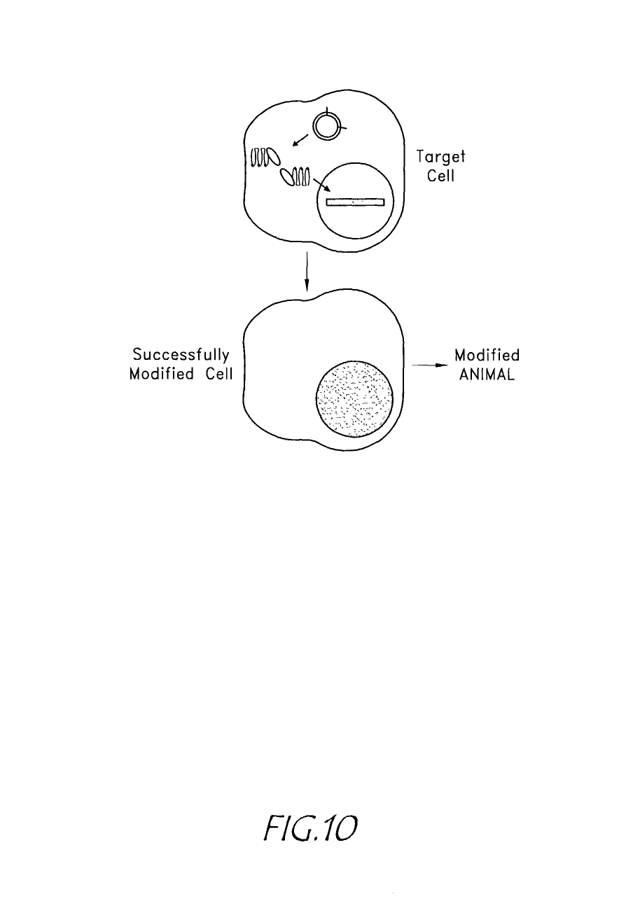

FIG. 10 summarizes one general method for modifying a cow cell and obtaining a modified cow. A recombination vector containing the target gene associated with mad cow disease or a portion thereof in which the coding sequence has been disrupted is introduced into the cow cell so that it can recombine by homologous recombination with genomic DNA. In some embodiments, the vector is introduced at a concentration of about 100 ng/μl, but any concentration which is sufficient to permit homologous recombination may be used. The homologous recombination construct containing the disrupted coding sequence is either introduced into the cell alone or with a ZFE protein by microinjection or using techniques such as lipofection or calcium phosphate transfection, for example.

ZFE protein from Example 1E is delivered such as by microinjection, for example, into a primary cow cell either alone or concurrently with the vector. The ZFE enhances homologous recombination between the vector and cellular genomic DNA. A range of concentrations of ZFE protein is injected. In some embodiments, this range is approximately 5-10 mg of protein per ml of buffer injected, but any concentration of ZFE which is sufficient to enhance the frequency of homologous recombination may be used. The DNA and the ZFE protein are resuspended in a buffer such as 10 mM Hepes buffer (pH 7.0) which contains 30 mM KCl.

Homologous recombination is the exchange of homologous stretches of DNA. In order to alter the genome by homologous recombination, DNA constructs containing areas of homology to genomic DNA are added to a cell. One challenge associated with homologous recombination is that it normally occurs rarely. A second problem is that there is a relatively high rate of random integration into the genome. (Capecchi, “Altering the genome by homologous recombination,” Science 244 (4910):1288-1292 (1989); the disclosure of which is hereby incorporated by reference in its entirety). The inclusion of ZFEs increases the rate of homologous recombination while the rate of random integration is unaffected.

Once the ZFE protein and the vector are delivered into the cell and homologous recombination occurs, a successfully modified cell is identified as described herein. The successfully modified cell is used to obtain a modified animal as described elsewhere herein.

### EXAMPLE 1G

### Generation of a PRNP Knockout

In order to knock out a gene associated with reduced susceptibility to mad cow disease, for example PRNP, the following reagents are constructed: two positive/negative DNA homologous recombination DNA constructs (one for each allele), two PRNP specific ZFEs and RAD51 protein. The positive negative constructs contain a fragment of the cow genomic DNA that flanks exon 3 of PRNP. Exon 3 is the only coding exon of PRNP. In one positive/negative construct EGFP under the control of a CMV promotor replaces Exon 3, utilizing restriction sites that flank the Exon. In the other positive/negative construct DsRed2 under the control of a CMV promotor replaces Exon 3, utilizing the same restriction sites that flank the Exon. Both the EGFP and DsRed2 “positive” markers are flanked by Lox P sites. At one end of the construct the coding region for human CD8 alpha chain under the control of a CMV promoter has been added as the “negative” marker. In combination, both ZFEs cut the cow genome only once at a sequence that lies within Exon 3 of PRNP. Bovine Rad51 was cloned from the pcDNA Cow cDNA library and may be used to enhance general recombination.

The cell in which PRNP is targeted is a Bovine embryonic fibroblast. This is the cell type from which most cows have been cloned by nuclear transfer. For example, C. Kubota, H. Yamakuchi, J. Todoroki, K. Mizoshita, N. Tabara, M. Barber, and X. Yang. Six cloned calves produced from adult fibroblast cells after long-term culture. Proc.Natl.Acad.Sci.U.S.A 97 (3):990-995, 2000.

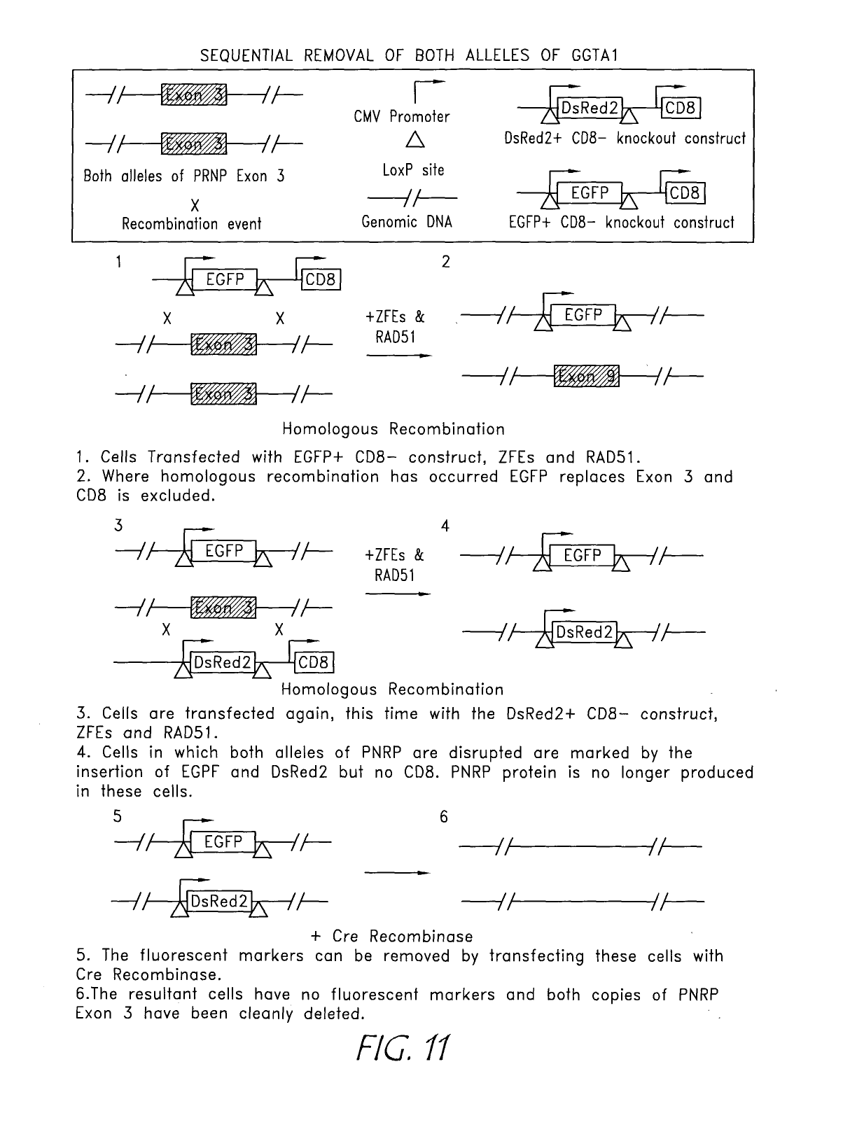

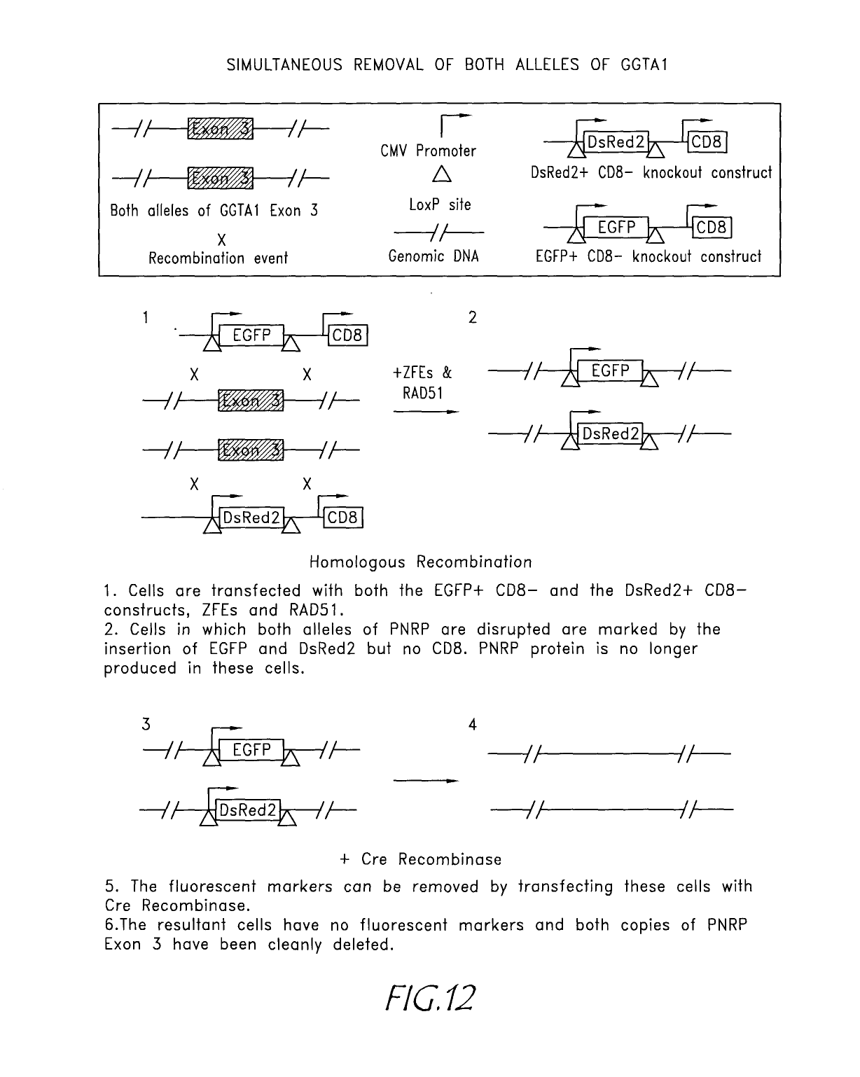

Both alleles of PRNP are either knocked out sequentially or simultaneously. Each method will be described in turn. These are illustrated in FIGS. 11 and 12, for example, and described in detail below.

The sequential method proceeds in the following way. Firstly, the construct containing EGFP positive marker and CD8 negative marker is introduced into cow embryonic fibroblasts using Fugene 6 (Roche). At the same time the two PRNP specific ZFEs and bovine RAD51 are introduced using chemicals like Chariot™ (Active Motif) or BioPorter™ (Gene Therapy Systems, Inc.). After a period of 48 to 72 hours these cells are labeled with an anti-CD8 antibody fluorescently labeled with APC (eBioscience). APC can be detected by FACS analysis as a colour distinct from both EGFP and DsRed2 which are in turn distinct from one another.

By FACS analysis some cells will not produce any color. In these cells there has been no recombination with the introduced DNA. Other cells will produce colour from both EGFP and anti-CD8 APC. In these cells random integration has occurred. The last group of cells will only produce colour from EGFP. In this group of cells it is likely that homologous recombination will have occurred. These cells will be single cell sorted away from the other cells.

Individual EGFP+ cells are cultured in a 96 well tissue culture plate with the appropriate media and feeder cells necessary for viability. The feeder cells will have been previously irradiated so that they cannot divide. Once the wells have divided for a period of one to two weeks there will be between 256 and 65536 cells. Genomic DNA is prepared from half of these cells. PCR is performed to check that EGFP has integrated in the expected position in the genome.

Clones of cells identified in this way are expanded in tissue culture for a further week until there are approximately 5 million cells. A portion of these cells are frozen down at this point. The remaining cells have the construct containing the DsRed2 positive marker and CD8 negative marker introduced using Fugene 6 (Roche). At the same time the two PRNP specific ZFEs and porcine RAD51 are again introduced using chemicals like Chariot™ (Active Motif) or BioPorter™ (Gene Therapy Systems, Inc.). After a period of 48 to 72 hours these cells are labeled with an anti-CD8 antibody fluorescently labeled with APC (eBioscience).

By FACS analysis some cells only produce colour from EGFP. In these cells there has been no further recombination with the introduced DNA. Other cells produce color from EGFP, DsRed2 and anti-CD8 APC. In these cells random integration has occurred. The last group of cells produce colour from EGFP and DsRed2 only. In this group of cells, it is likely that homologous recombination will have occurred once more in these cells. These cells will be single cell sorted away from the other cells.

Individual cells which are positive for both are cultured in a 96 well tissue culture plate with the appropriate media and feeder cells necessary for viability. The feeder cells will have been previously irradiated so that they cannot divide. Once the wells have divided for a period of one to two weeks there will be between 256 and 65536 cells. Genomic DNA is prepared from half of these cells. Two PCR reactions are performed to check that both the EGFP and the DsRed2 have integrated in the expected position in the genome. As a further control to check that both alleles of PRNP have been knocked out in these cells they are labeled with an anti-PmP antibody (Chemicon International, Temecula Calif.) fluorescently labeled with APC. Cells in which both alleles of GGTA1 have been disrupted produce color from EGFP and DsRed2 but not from the anti-PrnP APC labeled antibody. A portion of these cells are frozen down at this point.

The remaining cells are expanded in culture for a period of one to two weeks. The Cre recombinase protein will then be introduced using chemicals like Chariot™ (Active Motif) or BioPorter™ (Gene Therapy Systems, Inc.). In a proportion of these cells recombination will occur between the LoxP sites that flank the EGFP and the DsRed2 markers, excising both of these marker genes. These cells are labeled with an anti-PrnP antibody fluorescently labeled with APC. FACs analysis is used to sort out cells that do not produce any colour from any of EGFP, DsRed2 and APC labeled anti-PrnP. These cells are checked for viability, normal chromosome compliment and any that appear normal are either directly frozen down or used to produce PRNP null cows by nuclear transfer.

The simultaneous removal of both alleles of PRNP proceeds in the following way. The embryonic cow fibroblast will have the constructs containing both the EGFP positive marker and the CD8 negative marker as well as ones with the DsRed2 positive marker and CD8 negative marker introduced using Fugene 6 (Roche). At the same time the two PRNP specific ZFEs and porcine RAD51 are introduced using chemicals like Chariot™ (Active Motif) or BioPorter™ (Gene Therapy Systems, Inc.). After a period of 48 to 72 hours these cells are labeled with an anti-CD8 antibody fluorescently labeled with APC (eBioscience).

By FACS analysis some cells will only produce no colour. In these cells there has been no recombination with the introduced DNA. Other cells produce colour from EGFP and anti-CD8 APC and/or DsRed and anti-CD8 APC. In these cells random integration has occurred of one or both constructs. The last group of cells produces colour from EGFP and DsRed2 only. In this group of cells, it is likely that homologous recombination has occurred at both alleles. These cells are single cell sorted away from the other cells.

Individual cells which are positive for both are cultured in a 96 well tissue culture plate with the appropriate media and feeder cells necessary for viability. The feeder cells have been previously irradiated so that they cannot divide. Once the wells have divided for a period of one to two weeks there will be between 256 and 65536 cells. Genomic DNA is prepared from half of these cells. Two PCR reactions are performed to check that both the EGFP and the DsRed2 have integrated in the expected position in the genome. As a further control to check that both alleles of PRNP have been knocked out in these cells they are labeled with an anti-PrnP antibody fluorescently labeled with APC. Cells in which both alleles of PRNP have been disrupted produce colour from EGFP and DsRed2, but not from the anti-PrnP APC labeled antibody. A portion of these cells is frozen down at this point.

The remaining cells are expanded in culture for a period of one to two weeks. The Cre recombinase protein is then introduced using chemicals like Chariot™ (Active Motif) or BioPorter™ (Gene Therapy Systems, Inc.). In a proportion of these cells recombination occurs between the LoxP sites that flank the EGFP and the DsRed2 markers, excising both of these marker genes. These cells are labeled with an anti-PrnP antibody fluorescently labeled with APC. FACs analysis is used to sort out cells that do not produce any colour from any of EGFP, DsRed2 and APC labeled anti-PrnP. These cells are checked for viability, normal chromosome compliment and any that appear normal will either be directly frozen down of used to produce PRNP null cows by nuclear transfer.

### EXAMPLE 1H

### Obtaining a Cow Cell with a Modified Gene Associated with Mad Cow Disease

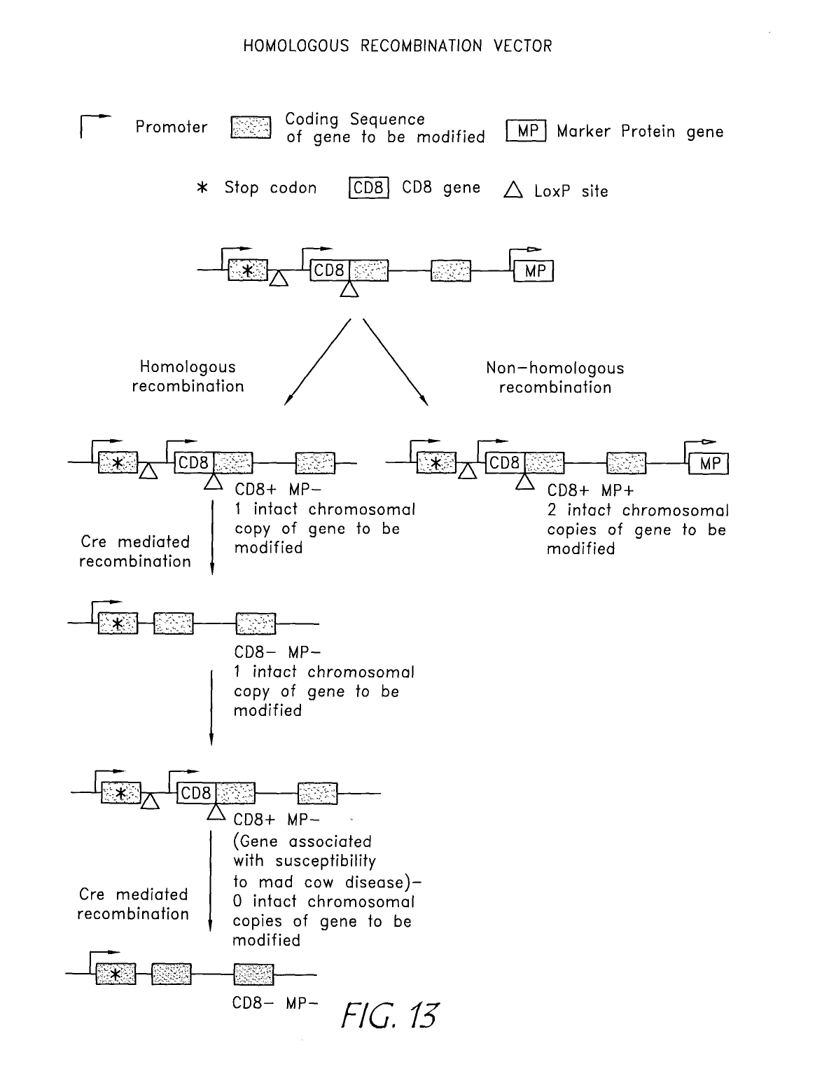

FIG. 13 illustrates one example of a vector and method for use in obtaining cow cells in which a gene associated with made cow disease has been modified. The modified gene or portion thereof or gene or portion thereof from a species other than cow may be introduced into the vector illustrated in FIG. 1 or the vector described in Capecchi, 1989, Science 244(4910):1288-1292.

The homologous recombination construct containing the modified coding sequence or coding sequence from a species other than cow is introduced into the cell using techniques such as lipofection, calcium phosphate transfection, electroporation, microinjection, or any other method or reagent. With regard to microinjection, somatic cells or any other suitable cell may be used. (For example, Klein, C. and Raab-Traub, “Human neonatal lymphocytes immortalized after micro injection of Epstein-Barr virus DNA,” J. Virol. 61:1552-1558 (1987)). Also, techniques have been developed for injection of adherent cells using very thin needles. (Davis, B. R. et al., “Glass needle mediated microinjection of macromolecules and transgenes into primary human blood stem/progenitor cells,” Gene Ther. 95:437 (2000)).

The skilled artisan will appreciate that any suitable cell type can used for the genetic manipulation. For example, as mentioned above, somatic cells can be used. Examples of cell types that have been successfully used in genetic engineering include, primary skin fibroblasts (Kubota, C. et al., “Six cloned calves produced from adult fibroblast cells after long-term culture,” Proc. Natl. Acad. Sci. USA 97:990-5 (2000)); bovine granulosa cells (Polejaeva, I. A. et al., “Cloned pigs produced by nuclear transfer from adult somatic cells,” Nature 407:86-90 (2000)); bovine fetal fibroblasts (Schnieke, A. E. et al., “Human factor IX transgenic sheep produced by transfer of nuclei from transfected fetal fibroblasts,” Science 278:2130-3 (1997)); and any other suitable cell type. The disclosures of each of the above references are hereby incorporated by reference in their entireties.

As illustrated in FIG. 13, the homologous recombination vector may comprise a gene associated with susceptibility to mad cow disease which has been modified by the creation of a stop codon in the coding sequence. The vector also includes a promoter operably linked to a nucleic acid encoding CD8 as a reporter gene and a promoter operably linked to a nucleic acid encoding a marker gene. For example, the marker gene can be the gene encoding green fluorescent protein (GFP). It will be appreciated that if it is desired to introduce a deletion, mutation resulting in one or more amino acid replacements, or gene from a species other than cow into the chromosome, the vector of FIG. 1 contains the desired sequence rather than the sequence bearing a stop codon.

As illustrated in FIG. 13, cells in which a homologous recombination event has occurred will be CD8⁺ and MP⁻, while cells in which the vector has integrated in a random location will be CD8⁺ and MP⁺. Accordingly, by performing several rounds of FACS separation using commercially available fluorescent antibodies against CD8 and the fluorescence of the marker protein, for example, cells in which a homologous recombination event has occurred may be separated from cells in which the vector has integrated randomly. The cells in which a homologous recombination event has occurred will contain one modified chromosomal copy of the gene associated with susceptibility to mad cow disease (i.e. the gene at which the homologous recombination event has occurred) and one intact chromosomal copy of the gene.

Cre mediated recombination between the LoxP sites is then allowed to occur in the cells in which the modified gene has been incorporated into the genome through homologous recombination. Cre may be provided in the cells in which a homologous recombination event has occurred by introducing Cre into the cells through lipofection (Baubonis et al., 1993, Nucleic Acids Res. 21:2025-9, the disclosure of which is incorporated herein by reference in its entirety), or by transfecting the cells with a vector comprising an inducible promoter operably linked to a nucleic acid encoding Cre (Gu et al., 1994, Science 265:103-106, the disclosure of which is incorporated herein by reference in its entirety). Cells in which Cre mediated recombination has occurred will be CD8⁻ and can be separated from CD8⁺ cells in which Cre mediated recombination has not occurred by performing several rounds of FACS.

If desired, the chromosomal structure of the separated cells may be verified by amplifying the target gene using PCR and sequencing the resulting amplicons to confirm the presence of one intact copy of the gene and one modified copy. Alternatively, the chromosomal structure of the separated cells may be verified by performing a Southern blot.

The remaining intact copy of the gene encoding a cow prion gene is then disrupted as follows. The homologous recombination vector is introduced into the cells comprising one intact copy of the gene and one modified copy of the gene. Cells in which homologous recombination has occurred at the formerly intact copy of the gene are identified by separating CD8⁺MP⁻ cells from CD8+MP⁺ cells by FACS as described above. In addition, if the cells normally expressed the target gene, fluorescent antibodies against the gene associated with mad cow disease may be used in a FACS procedure to separate cells which do not bind the antibody (i.e. cells in which both copies of the gene have been disrupted) from cells which bind the antibody (i.e. cells in which one copy of the gene is intact). Antibody may be obtained by methods well known to those of skill in the art.

Another round of Cre mediated recombination is allowed to occur to delete the CD8 gene in the cells.

If desired, the chromosomal structure of the separated cells may be verified by amplifying the target gene using PCR and sequencing the resulting amplicons to confirm the presence of two modified copies of the target gene. Alternatively, the chromosomal structure of the separated cells may be verified by performing a Southern blot.

FIG. 13 summarizes the above procedures.

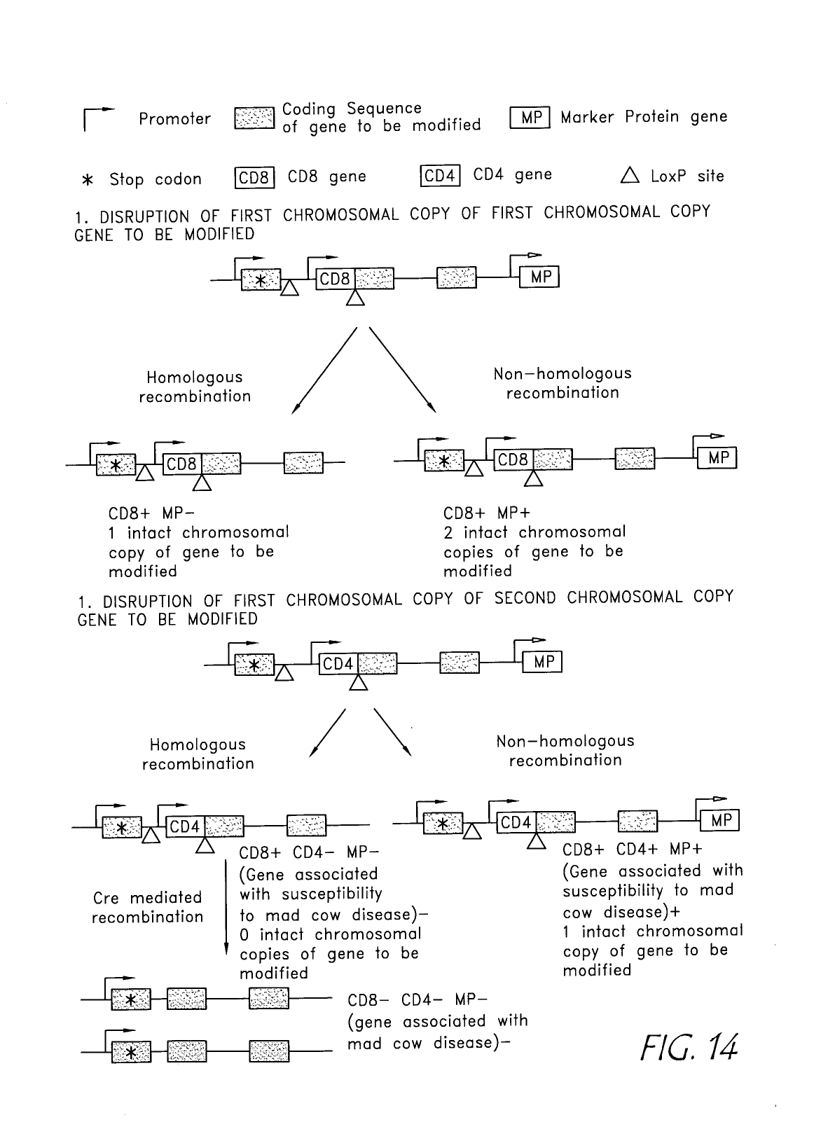

Alternatively, cells in which both chromosomal copies of a gene associated with susceptibility to mad cow disease are modified may be obtained as follows. The first chromosomal copy of the target gene is modified as described above. As described above, a first homologous recombination vector comprising a gene associated with susceptibility to mad cow disease, which has been modified by the creation of a stop codon in the coding sequence is introduced into the cell. The vector also includes a promoter operably linked to a nucleic acid encoding CD8 as a reporter gene and a promoter operably linked to a nucleic acid encoding a marker protein (MP), such as for example, green fluorescent protein (GFP). Again, it will be appreciated that if it is desired to introduce a deletion, mutation resulting in one or more amino acid replacements, or gene from a species other than cow into the chromosome, the first homologous recombination vector of FIG. 14 contains the desired sequence rather than the sequence bearing a stop codon.

As illustrated in FIG. 14, cells in which a homologous recombination event has occurred will be CD8⁺ and MP⁻, while cells in which the vector has integrated in a random location will be CD8⁺ and MP⁺. Accordingly, by performing several rounds of FACS separation using commercially available fluorescent antibodies against CD8 and the fluorescence of the MP, such as GFP, for example, cells in which a homologous recombination event has occurred may be separated from cells in which the vector has integrated randomly. As illustrated in FIG. 14, the cells in which a homologous recombination event has occurred will contain one modified chromosomal copy of the gene associated with susceptibility to mad cow disease (i.e. the gene at which the homologous recombination event has occurred) and one intact chromosomal copy of the gene.

If desired, the chromosomal structure of the separated cells may be verified by amplifying the target gene using PCR and sequencing the resulting amplicons to confirm the presence of one intact copy of the gene and one modified copy. Alternatively, the chromosomal structure of the separated cells may be verified by performing a Southern blot.

A second homologous recombination vector is then introduced into the cells in which one chromosomal copy of the target gene has been disrupted. The second homologous recombination vector is similar to the one used to disrupt the first chromosomal copy of the target gene except that rather than containing a gene encoding the CD8 protein operably linked to a promoter, the second homologous recombination vector contains a gene encoding the CD4 protein operably linked to a promoter. As illustrated in FIG. 14, cells in which the second homologous recombination vector has integrated into the chromosome through a homologous recombination event occurred will be CD4⁺ and MP⁻, while cells in which the vector has integrated in a random location will be CD4⁺ and MP⁺. Accordingly, by performing several rounds of FACS separation using commercially available fluorescent antibodies against CD4 and the fluorescence of MP, such as GFP, for example, cells in which a homologous recombination event has occurred may be separated from cells in which the vector has integrated randomly. If desired, the FACS analysis may also use antibody against CD8, since the cells will be CD8⁺ by virtue of the chromosomal integration of the first homologous recombination vector through a homologous recombination event. In addition, if the cells normally expressed the target gene, fluorescent antibodies against the gene associated with susceptibility to mad cow disease may be used in a FACS procedure to separate cells which do not bind the antibody (i.e. cells in which both copies of the gene have been disrupted) from cells which bind the antibody (i.e. cells in which one copy of the gene is intact). Antibody against the polypeptide encoded by the gene associated with susceptibility to mad cow disease may be by methods well known to those of skill in the art. As illustrated in FIG. 14, the cells in which the second homologous recombination event has occurred at the second chromosomal copy of the target gene will have both chromosomal copies of the target gene modified.

Cre mediated recombination between the LoxP sites is then allowed to occur in the cells in which the both chromosomal copies of the target gene have been modified. Cells in which Cre mediated recombination has occurred in both of the integrated vectors will be CD8⁻ and CD4⁻ and can be separated from cells in which Cre mediated recombination has not occurred in both of the integrated vectors (which will be CD8⁺CD4⁺, CD8⁺CD4⁻, or CD8⁻CD4⁺ depending on whether Cre mediated recombination has not occurred at all or whether it occurred in one of the two integrated vectors) by performing several rounds of FACS. In addition, if the cells normally expressed the target gene, fluorescent antibodies against the gene associated with susceptibility to mad cow disease may be used in a FACS procedure to separate cells which do not bind the antibody (i.e. cells in which both copies of the gene have been disrupted) from cells which bind the antibody (i.e. cells in which one copy of the gene is intact). Antibody against the polypeptide encoded by the gene associated with susceptibility to mad cow disease may be obtained by methods well known to those of skill in the art.

If desired, the chromosomal structure of the separated cells may be verified by amplifying the target gene using PCR and sequencing the resulting amplicons to confirm the presence of two modified copies of the target gene. Alternatively, the chromosomal structure of the separated cells may be verified by performing a Southern blot.

FIG. 14 summarizes the above procedures.

It will be appreciated that, if desired, the homologous recombination vector used to modify the first chromosomal copy of the target gene may be the vector which contains the CD4 gene and the homologous recombination vector used to modify the second chromosomal copy of the target gene may be the vector which contains the CD8 gene. The skilled artisan will appreciate that CD4 and CD8 are examples of what can be used. Any other suitable polypeptide and gene can be used.

If desired, the structure of the targeted genes in the cells obtained by FACS analysis may be evaluated by performing a Southern blot or PCR analysis to confirm that the both copies of the targeted genes have been disrupted.

### EXAMPLE 2

### Testing the Genetically Modified Cells for Susceptibility to Prion Disease

When the cells have been genetically modified, i.e., both alleles of the prion gene have been deleted, the cells are infected with the prion protein. Theoretically, there should be no chance of infecting these cells since the gene encoding for the prion protein is deleted. Different strains are tested to ensure these cells are resistant to infection. Infected cells are lysed and are subjected to separation by SDS-PAGE electrophoresis and Western blot. The blot is probed by an antibody recognizing the pathological isoform of the prion protein to verify if there is any injectable protein still present. A positive control is unmodified cells, which should still be susceptible to infection. (Birkett, C. R. et al., “Scrapie strains maintain biological phenotypes on propagation in a cell line in culture,” EMBO 20:3351-58 (2001), the disclosure of which is hereby incorporated by reference in its entirety).

### EXAMPLE 3

### Generation of Animals Comprising Cells with Modified Genes

After cells having a modified gene associated with susceptibility to mad cow disease are generated as described above, they are used to generate genetically modified cows. Nuclear transfer using nuclei from cells having modified genes associated with susceptibility to mad cow disease is performed as described by Wilmut et al., 1997, Nature. 385(6619)810-813, U.S. Pat. No. 6,147,276, U.S. Pat. No. 5,945,577 or U.S. Pat. No. 6,077,710, the disclosures of which are incorporated herein by reference in their entireties Briefly, the nuclei are transferred into enucleated fertilized oocytes. A large number of oocytes are generated in this manner. Approximately ten animals are fertilized with the oocytes, with at least six fertilized embryos being implanted into each animal and allowed to progress through birth. They are bred cattle onto a large population to avoid inbreeding.

Animals comprising cells in which a genes associated with susceptibility to mad cow disease have been modified may also be generated using other methods. For example, as discussed above, stem cell-based technologies may be employed.

### EXAMPLE 4

### Products and Compositions from the Cows

Various products and compositions are obtained from the cows having reduced susceptibility to mad cow disease by methods familiar to those of skill in the art. Meat is obtained from the cows. The meat is packaged using conventional methods. The meat can be used by humans and/or by other animals. In addition, growth hormone, blood components, such as hemoglobin, bovine serum, fetal calf serum, other proteins needed for research in large quantities, gelatin, collagen, human proteins or other proteins expressed by the cows through genetic engineering, or other desired components of the cow may be obtained using conventional methods.

For example, bovine serum or fetal calf serum obtained from cows having reduced susceptibility to mad cow disease may be used for growing tissue culture cells which produce products to be consumed or used by humans, such as therapeutic proteins, vectors to be used for gene therapy or vaccination, or viral vaccines, thereby reducing the risk of inducing variant Crutzfeldt-Jakob Disease in the humans. Similarly, the other products and compositions also carry a reduced risk of inducing disease in a human recipient that comes in contact with them or with materials derived from them.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims. All documents cited herein are incorporated herein by reference in their entirety.

What is claimed is:

1. A genetically engineered cow cell in which a gene associated with mad cow disease has been modified to provide reduced susceptibility to mad cow disease.

2. The genetically engineered cell of claim 1, wherein said cell is selected from the group consisting of a skin fibroblast, a granulosa cell, a stem cell, a germ cell, a fetal fibroblast and a fetal cell.

3. The genetically engineered cell of claim 1, wherein both chromosomal copies of said gene or portions thereof have been replaced with a homologous sequence which provides reduced susceptibility to mad cow disease.

4. The genetically engineered cell of claim 3, wherein said homologous sequence comprises a modified version of a gene which encodes an mRNA corresponding to the cDNA nucleotide sequence of SEQ ID NO: 1 or a portion of such a gene wherein said gene or portion thereof contains a stop codon in the open reading frame which encodes the polypeptide of SEQ ID NO: 2.

5. The genetically engineered cell of claim 3, wherein said homologous sequence comprises a modified version of a gene which encodes an mRNA corresponding to the cDNA nucleotide sequence of SEQ ID NO: 1 or a portion of such a gene wherein said gene or portion thereof contains a deletion therein.

6. The genetically engineered cell of claim 3, wherein said gene encodes a polypeptide comprising the amino acid sequence of SEQ ID NO: 2.

7. The genetically engineered cell of claim 6, wherein one or more amino acids of SEQ ID NO: 2 is replaced with a different amino acid.

8. The genetically engineered cell of claim 7, wherein the methionine at position 129 of SEQ ID NO: 2 is replaced with an amino acid other than methionine.

9. The recombinant cell of claim 7, wherein said methionine at position 129 is replaced by an amino acid selected from the group consisting of alanine, valine, cysteine, isoleucine, leucine, phenylalanine, tryptophane and tyrosine.

10. The genetically engineered cell of claim 3, wherein said homologous sequence comprises a modified version of a cow prion gene which contains a stop codon in the open reading frame which encodes the cow prion polypeptide.

11. The genetically engineered cell of claim 3, wherein said homologous sequence comprises a modified version of a cow prion gene which contains a deletion therein.

12. The genetically engineered cell of claim 3, wherein said gene encodes a polypeptide comprising the amino acid sequence of a cow prion polypeptide.

13. The genetically engineered cell of claim 3, wherein said modified cell is generated by replacing the gene encoding the cow prion protein or a portion thereof with a gene encoding a prion protein from a species other than cow or a portion thereof.

14. The genetically engineered cell of claim 13, wherein said species is selected from the group consisting of a sheep, a goat, a marsupial, and a mouse.

15. The genetically engineered cell of claim 3, wherein at least one allele of said gene associated with mad cow disease has been replaced with an allele that is resistant to mad cow disease.

16. The genetically engineered cell of claim 3, wherein at least one allele has been modified to include one or more point mutations which alter the identity of one or more amino acids.

17. A genetically modified cow generated from the recombinant cell of claim 1.

18. A recombinant nucleic acid comprising a 5′ region homologous to a portion of a gene associated with susceptibility to mad cow disease, a 3′ region homologous to a portion of a gene associated with susceptibility to mad cow disease, and at least a portion of the coding sequence of said gene disposed between said 5′ region and said 3′ region, said at least a portion of the coding sequence containing a sequence therein which reduces susceptibility to mad cow disease.

19. The recombinant nucleic acid of claim 18, wherein said sequence comprises a modified version of a gene which encodes an mRNA corresponding to the cDNA nucleotide sequence of SEQ ID NO: 1 or a portion of such a gene wherein said gene or portion thereof contains a stop codon in the open reading frame which encodes the polypeptide of SEQ ID NO: 2.

20. The recombinant nucleic acid of claim 18, wherein said sequence comprises a modified version of a gene which encodes an mRNA corresponding to the cDNA nucleotide sequence of SEQ ID NO: 1 or a portion of such a gene wherein said gene or portion thereof contains a deletion therein.

21. The recombinant nucleic acid of claim 18, wherein said gene encodes a polypeptide comprising the amino acid sequence of SEQ ID NO: 2.

22. The recombinant nucleic acid of claim 21, wherein said gene encodes a polypeptide comprising one or more point mutations or replacement amino acids.

23. The recombinant nucleic acid of claim 22, wherein the methionine at position 129 of SEQ ID NO: 2 is replaced with an amino acid other than methionine.

24. The recombinant nucleic acid of claim 23, wherein said methionine at position 129 is replaced by an amino acid selected from the group consisting of alanine, valine, cysteine, isoleucine, leucine, phenylalanine, tryptophane and tyrosine.

25. The recombinant nucleic acid of claim 18, wherein said sequence comprises a modified version of a cow prion gene which contains a stop codon in the open reading frame which encodes the cow prion polypeptide.

26. The recombinant nucleic acid of claim 18, wherein said sequence comprises a modified version of a cow prion gene which contains a deletion therein.

27. The recombinant nucleic acid of claim 18, wherein said gene encodes a polypeptide comprising the amino acid sequence of a cow prion polypeptide.

28. The recombinant nucleic acid of claim 18, wherein said sequence comprises a gene encoding a prion protein from a species other than cow.

29. The recombinant nucleic acid of claim 28, wherein said species is selected from the group consisting of a sheep, a goat, a marsupial, and a mouse.

30. The recombinant nucleic acid of claim 18 further comprising at least one nucleic acid encoding a detectable polypeptide, said at least one nucleic acid being operably linked to a promoter.

31. The recombinant nucleic acid of claim 30, wherein said detectable polypeptide is selected from the group consisting of CD8, CD4 and green fluorescent protein.

32. The recombinant nucleic acid of claim 30, wherein said recombinant nucleic acid comprises a nucleic acid encoding CD8 or CD4 operably linked to a promoter and a nucleic acid encoding green fluorescent protein operably linked to a promoter.

33. The recombinant nucleic acid of claim 18, wherein at least one nucleic acid encoding a detectable polypeptide is flanked by a site which facilitates recombination.

34. The recombinant nucleic acid of claim 33, wherein said site which facilitates recombination is a Lox P site

35. A method of modifying a gene associated with susceptibility to mad cow disease comprising:
introducing a nucleic acid comprising a sequence homologous to at least a portion of the coding region of said gene into a cow cell, wherein said homologous sequence comprises a sequence which reduces susceptibility to mad cow disease; and
replacing at least one chromosomal copy of said gene with said homologous sequence.

36. The method of claim 35, wherein said sequence comprises a modified version of a gene which encodes an mRNA corresponding to the cDNA nucleotide sequence of SEQ ID NO: 1 or a portion of such a modified gene wherein said gene or portion thereof contains a stop codon in the open reading frame which encodes the polypeptide of SEQ ID NO: 2.

37. The method of claim 35, wherein said sequence comprises a modified version of a gene which encodes an mRNA corresponding to the cDNA nucleotide sequence of SEQ ID NO: 1 or a portion of such a gene wherein said modified gene or portion thereof contains a deletion therein.

38. The method of claim 35, wherein said gene encodes a polypeptide comprising the amino acid sequence of SEQ ID NO: 2.

39. The method of claim 38, wherein the methionine at position 129 of SEQ ID NO: 2 is replaced with an amino acid other than methionine.

40. The method of claim 39, wherein said methionine at position 129 is replaced by an amino acid selected from the group consisting of alanine, valine, cysteine, isoleucine, leucine, phenylalanine, tryptophane and tyrosine.

41. The method of claim 35, wherein said sequence comprises a modified version of a cow prion gene which contains a stop codon in the open reading frame which encodes the polypeptide of the cow prion polypeptide.

42. The method of claim 35, wherein said sequence comprises a modified version of a cow prion gene which contains a deletion therein.

43. The method of claim 35, wherein said gene encodes a polypeptide comprising the amino acid sequence of a cow prion polypeptide.

44. The method of claim 35, wherein said sequence comprises a gene encoding a prion protein from a species other than cow.

45. The method of claim 44, wherein said species is selected from the group consisting of a sheep, a goat, a marsupial, and a mouse.

46. The method of claim 35, further comprising enhancing the rate of recombination by introducing a double stranded break in said nucleic acid in a region in the vicinity of the gene associated with susceptibility to mad cow disease.

47. The method of claim 46, wherein said double stranded break is introduced using at least one zinc finger endonuclease protein.

48. The method of claim 35, further comprising introducing a substance that enhances the rate of homologous recombination in said cell.

49. The method of claim 48, wherein said substance is RAD51 or RAD52.

50. A method of obtaining a cow with reduced susceptibility to mad cow disease comprising:
obtaining a cell from a cow;
generating a modified cell by modifying a gene in said cell which is associated with mad cow disease such that said modified gene provides reduced susceptibility to mad cow disease relative to an unmodified gene; and
generating a cow from said modified cell, wherein said cow comprises cells in which said gene associated with susceptibility to mad cow disease has been modified.

51. The method of claim 50, wherein said cell is a somatic cell.

52. The method of claim 51, wherein said somatic cell is selected from the group consisting of a fibroblast, a granulosa cell, and fetal fibroblast.

53. The method of claim 50 where in said cell is selected from the group consisting of a germ cell, a stem cell, a fetal fibroblast, and a fetal cell.

54. The method of claim 50, wherein said gene is modified by replacing both chromosomal copies of said gene or a portion thereof with a homologous sequence which provides reduced susceptibility to mad cow disease.

55. The method of claim 54, wherein said homologous sequence comprises a modified version of a gene which encodes an mRNA corresponding to the cDNA nucleotide sequence of SEQ ID NO: 1 or a portion of such a gene wherein said gene or portion thereof contains a stop codon in the open reading frame which encodes the polypeptide of SEQ ID NO: 2.

56. The method of claim 54, wherein said homologous sequence comprises a modified version of a gene which encodes an mRNA corresponding to the cDNA nucleotide sequence of SEQ ID NO: 50 or a portion of such a gene wherein said gene or portion thereof contains a deletion therein.

57. The method of claim 54, wherein said gene encodes a polypeptide comprising the amino acid sequence of SEQ ID NO: 2.

58. The method of claim 57 wherein the methionine at position 129 of SEQ ID NO: 2 is replaced with an amino acid other than methionine.

59. The method of claim 58, wherein said methionine at position 129 is replaced by an amino acid selected from the group consisting of alanine, valine, cysteine, isoleucine, leucine, phenylalanine, tryptophane and tyrosine.

60. The method of claim 54, wherein said homologous sequence comprises a modified version of a cow prion gene which contains a stop codon in the open reading frame which encodes the cow prion polypeptide.

61. The method of claim 54, wherein said homologous sequence comprises a modified version of a cow prion gene which contains a deletion therein.

62. The method of claim 54 wherein said gene encodes a polypeptide comprising the amino acid sequence of a cow prion polypeptide.

63. The method of claim 50, wherein said modified cell is generated by replacing the gene encoding the cow prion protein or a portion thereof with a gene encoding a prion protein from a species other than cow or a portion thereof.

64. The method of claim 63, wherein said species is selected from the group consisting of a sheep, a goat, a marsupial, and a mouse.

65. A cow in which a gene associated with mad cow disease or a portion thereof has been replaced with a sequence which reduces susceptibility to mad cow disease.

66. The cow of claim 65, wherein both chromosomal copies of said gene or a portion thereof have been replaced with a homologous sequence which provides reduced susceptibility to mad cow disease.

67. The cow of claim 66 wherein said homologous sequence comprises a modified version of a gene which encodes an mRNA corresponding to the cDNA nucleotide sequence of SEQ ID NO: 1 or a portion of such a gene wherein said gene or portion thereof contains a stop codon in the open reading frame which encodes the polypeptide of SEQ ID NO: 2.

68. The cow of claim 66, wherein said homologous sequence comprises a modified version of a gene which encodes an mRNA corresponding to the cDNA nucleotide sequence of SEQ ID NO: 1 or a portion of such a gene wherein said gene or portion thereof contains a deletion therein.

69. The cow of claim 66, wherein said gene encodes a polypeptide comprising the amino acid sequence of SEQ ID NO: 2.

70. The cow of claim 69, wherein the methionine at position 129 of SEQ ID NO: 2 is replaced with an amino acid other than methionine.

71. The cow of claim 70, wherein said methionine at position 129 is replaced by an amino acid selected from the group consisting of alanine, valine, cysteine, isoleucine, leucine, phenylalanine, tryptophane and tyrosine.

72. The cow of claim 66 wherein said homologous sequence comprises a modified version of a prion gene which contains a stop codon in the open reading frame which encodes the polypeptide of the cow prion polypeptide.

73. The cow of claim 66, wherein said homologous sequence comprises a modified version of a cow prion gene which contains a deletion therein.

74. The cow of claim 66, wherein said gene encodes a polypeptide comprising the amino acid sequence of cow prion polypeptide.

75. The cow of claim 66, wherein the gene encoding the cow prion protein or a portion thereof has been replaced with a gene encoding a prion protein from a species other than cow or a portion thereof.

76. The cow of claim 75, wherein said species is selected from the group consisting of a sheep, a goat, a marsupial and a mouse.

77. A composition comprising meat from a genetically modified cow according to claim 65.

78. A method of packaging meat comprising obtaining meat from a genetically modified cow according to claim 65 and packaging said meat in a packaging material.

79. A composition comprising bovine serum or fetal calf serum from a genetically modified cow according to claim 65.

80. A composition comprising one or more proteins from a genetically modified cow according to claim 65.
